(19) United States
(12) Patent Application Publication
(10) Pub. No.: US 2002/0076775 A1
(43) Pub. Date: Jun. 20, 2002

(54) WRKY transcription factors and methods of use

(76) Inventors: Virginia C. Crane, Des Moines, IA (US); Omolayo Famodu, Newark, DE (US); Xu Hu, Urbandale, IA (US); Guihua Lu, Urbandale, IA (US); Lingyu Zhang, Johnston, IA (US)

Correspondence Address: Pioneer Hi-Bred International, Inc., Corporate Intellectual Property, 7100 N.W. 62nd Avenue, P.O. Box 1000, Johnston, IA, 50131-1000, US

(21) Appl. No.: 09/810,264

(22) Filed: Mar. 16, 2001

Related U.S. Application Data
(60) Provisional application No. 60/190,467, filed on Mar. 17, 2000.

(51) Int. Cl. A01H 5/00; C12N 9/00; C12P 21/02; C12N 5/04

(52) U.S. Cl. 435/183; 435/410; 435/69.1; 800/279

(57) ABSTRACT

The invention provides isolated WRKY nucleic acids and their encoded proteins. The present invention provides methods and compositions relating to altering WRKY concentration and/or composition of plants. The present invention also relates to transcriptional regulatory regions of WRKY polynucleotides and their use to regulate heterologous gene expression. The invention further provides recombinant expression cassettes, host cells, and transgenic plants.

### CROSS REFERENCE TO RELATED APPLICATIONS

This application claims the benefit of U.S. provisional application No. 60/190,950, filed Mar. 21, 2000, which is herein incorporated by reference.

### BACKGROUND OF THE INVENTION

Plant disease outbreaks have resulted in catastrophic crop failures that have triggered famines and caused major social change. Generally, the best strategy for plant disease control is to use resistant cultivars selected or developed by plant breeders for this purpose. However, the potential for serious crop disease epidemics persists today, as evidenced by outbreaks of the Victoria blight of oats and southern corn leaf blight. Accordingly, molecular methods are needed to supplement traditional breeding methods to protect plants from pathogen attack.

A host of cellular processes enables plants to defend themselves from disease caused by pathogenic agents. These processes apparently form an integrated set of resistance mechanisms that is activated by initial infection and then limits further spread of the invading pathogenic microorganism.

WRKY proteins are a family of plant-specific zinc-finger-type factors implicated in the regulation of genes associated with a plant's response to a pathogen or stress, such as wounding. In addition, WRKY proteins have been implicated in senescence, trichome development and the biosynthesis of secondary metabolites. In parsley, WRKY proteins have been found to bind specifically to functionally defined TGAC-containing W box promoter elements within the Pathogenesis-Related Class 10 (PR-10) genes. The WRKY proteins in parsley are rapidly and locally activated in leaf tissue around the infection site of a pathogen. Transient expression studies in parsley protoplasts showed that a specific arrangement of W box elements in the WRKY1 promoter itself is necessary and sufficient for early activation and that WRKY1 binds to such elements (Rushton, et al., EMBO Journal, 15(2):5690-5700 (1996)).

WRKY proteins have been classified into three groups. Group I typically has two WRKY domains of a unique zinc-finger-like motif. Group II typically has only one WRKY domain. Group III has one WRKY domain but instead of the C₂-H₂ motif found in Groups I and II, the WRKY domain in Group III has a C₂-HC motif.

The present invention discloses WRKY polynucleotides from sunflower, maize, rice, wheat and soybean. WRKY polynucleotides may be used to engineer plants to resist pathogens and to survive stress. In addition, WRKY cDNA clones and DNA segments of genomic DNA, and their homologs and derivatives, may be used as molecular probes to track inheritance of corresponding loci in genetic crosses, and thus facilitate the plant breeding process. Moreover, these DNA sequences may also be used as probes to isolate, identify and genetically map WRKY and other closely related disease resistance genes. Further the polynucleotides of the present invention, either as a full-length or a sub-sequence, could be used to find genes and their promoters that respond to a WRKY domain.

The present invention also discloses a transcriptional regulatory region sequence from a sunflower WRKY gene, which can induce expression of a gene of interest during pathogen infection or in the presence of oxalic acid or salicylic acid. Gene expression encompasses a number of steps from DNA template to the final protein or protein product. Initiation of transcription of a gene is generally understood to be the predominant controlling factor in determining expression of a gene.

Controlling the expression of agronomic genes in transgenic plants is considered by those skilled in the art to provide several advantages over generalized or constitutive expression. The ability to control gene expression may be utilized to time expression for when a pathogen attacks a plant thus avoiding certain regulatory and commercial issues. A pathogen or chemically-inducible promoter can reduce potential yield loss by limiting expression of some pernicious, yet useful agronomic genes to only when it is needed. Further advantages of utilizing promoters that function in an inducible manner include reduced resource drain on the plant in making a gene product constitutively. Said gene products may include general toxin degradative genes such as oxalate oxidase or other disease resistance genes. There is a need in the art for novel promoters capable of driving pathogen or chemical-inducible gene expression in plants. It is considered important by those skilled in the art to continue to provide pathogen or chemical-inducible transcriptional regulatory regions capable of driving expression of genes that may confer a selective advantage to a plant.

### SUMMARY OF THE INVENTION

Generally, it is the object of the present invention to provide nucleic acids and proteins relating to WRKY. It is an object of the present invention to provide transgenic plants comprising the nucleic acids of the present invention. It is another object of the present invention to provide methods for modulating, in a transgenic plant, the expression of the nucleic acids of the present invention.

Therefore, in one aspect, the present invention relates to an isolated nucleic acid comprising a member selected from the group consisting of (a) a polynucleotide encoding a polypeptide of the present invention; (b) a polynucleotide having at least 75 or 80% sequence identity to the polynucleotides of the present invention; (c) a polynucleotide that hybridizes under high stringency conditions to the polynucleotides of the present invention; and (d) a polynucleotide complementary to a polynucleotide of (a) through (c). The isolated nucleic acid can be DNA. The isolated nucleic acid can also be RNA.

In another aspect, the present invention relates to vectors comprising the polynucleotides of the present invention. Also the present invention relates to recombinant expression cassettes, comprising a nucleic acid of the present invention operably linked to a promoter.

In another aspect, the present invention is directed to a host cell into which has been introduced the recombinant expression cassette.

In yet another aspect, the present invention relates to a transgenic plant or plant cell comprising a recombinant expression cassette with a promoter operably linked to any of the isolated nucleic acids of the present invention. Preferred plants containing the recombinant expression cassette of the present invention include but are not limited to maize, soybean, sunflower, sorghum, canola, wheat, alfalfa, cotton, rice barley, and millet. The present invention also provides transgenic seed from the transgenic plant.

In another aspect, the present invention relates to an isolated protein selected from the group consisting of (a) a polypeptide comprising at least 40 or 50 contiguous amino acids of a polypeptide of the present invention; (b) a polypeptide comprising at least 75 or 80% sequence identity to a polypeptide of the present invention; (c) a polypeptide encoded by a nucleic acid of the present invention; and (d) a polypeptide characterized by a polypeptide of the present invention.

In a further aspect, the present invention relates to a method of modulating the level of protein in a plant by introducing into a plant cell a recombinant expression cassette comprising a polynucleotide of the present invention operably linked to a promoter; culturing the plant cell under plant growing conditions to produce a regenerated plant; and inducing expression of the polynucleotide for a time sufficient to modulate the protein of the present invention in the plant. Preferred plants of the present invention include but are not limited to maize, soybean, sunflower, sorghum, canola, wheat, alfalfa, cotton, rice, barley, and millet. The level of protein in the plant can either be increased or decreased.

In addition, the present invention provides a transcriptional regulatory region capable of directing pathogen or chemical-induced gene expression. Further, the present invention provides for plants, plant cells, and seeds from the plant containing the transcriptional regulatory region. The present invention also provides for a method of expressing a heterologous nucleic acid during pathogen infection or upon chemical induction with the transcriptional regulatory region of the present invention.

### BRIEF DESCRIPTION OF THE SEQUENCE DESCRIPTIONS

The following sequence descriptions and sequence listings attached hereto comply with the rules governing nucleotide and/or amino acid sequence disclosures in patent applications as set forth in 37 C.F.R. §1.821-1.825.

SEQ ID NO: 1 is the nucleotide sequence comprising the maize ZmWRKY3-1 polynucleotide.

SEQ ID NO: 2 is the amino acid sequence of a maize ZmWRKY3-1 protein derived from the nucleotide sequence of SEQ ID NO: 1.

SEQ ID NOS: 3-8 are primer sequences used to isolate the sunflower WRKY polynucleotides.

SEQ ID NO: 9 is the nucleotide sequence comprising the sunflower SWRKY1-1 polynucleotide.

SEQ ID NO: 10 is the amino acid sequence of a sunflower SWRKY1-1 protein derived from the nucleotide sequence of SEQ ID NO: 9.

SEQ ID NO: 11 is the nucleotide sequence comprising the sunflower SWRKY1-2 polynucleotide.

SEQ ID NO: 12 is the amino acid sequence of a sunflower SWRKY1-2 protein derived from the nucleotide sequence of SEQ ID NO: 11.

SEQ ID NO: 13 is the nucleotide sequence comprising the sunflower SWRKY1-3 polynucleotide.

SEQ ID NO: 14 is the amino acid sequence of a sunflower SWRKY1-3 protein derived from the nucleotide sequence of SEQ ID NO: 13.

SEQ ID NO: 15 is the nucleotide sequence comprising the sunflower S WRKY1-4 polynucleotide.

SEQ ID NO: 16 is the amino acid sequence of a sunflower SWRKY1-4 protein derived from the nucleotide sequence of SEQ ID NO: 15.

SEQ ID NO: 17 is the nucleotide sequence comprising the rice WRKY1 polynucleotide.

SEQ ID NO: 18 is the amino acid sequence of a rice WRKY1 protein derived from the nucleotide sequence of SEQ ID NO: 17.

SEQ ID NO: 19 is the nucleotide sequence comprising the rice WRKY3 polynucleotide.

SEQ ID NO: 20 is the amino acid sequence of a rice WRKY3 protein derived from the nucleotide sequence of SEQ ID NO: 19.

SEQ ID NO: 21 is the nucleotide sequence comprising the soybean WRKY1 polynucleotide.

SEQ ID NO: 22 is the amino acid sequence of a soybean WRKY1 protein derived from the nucleotide sequence of SEQ ID NO: 21.

SEQ ID NO: 23 is the nucleotide sequence comprising the soybean WRKY2 polynucleotide.

SEQ ID NO: 24 is the amino acid sequence of a soybean WRKY2 protein derived from the nucleotide sequence of SEQ ID NO: 23.

SEQ ID NO: 25 is the nucleotide sequence comprising the soybean WRKY3 polynucleotide.

SEQ ID NO: 26 is the amino acid sequence of a soybean WRKY3 protein derived from the nucleotide sequence of SEQ ID NO: 25.

SEQ ID NO: 27 is the nucleotide sequence comprising the wheat WRKY2 polynucleotide.

SEQ ID NO: 28 is the amino acid sequence of a wheat WRKY2 protein derived from the nucleotide sequence of SEQ ID NO: 27.

SEQ ID NO: 29 is the nucleotide sequence comprising the wheat WRKY3 polynucleotide.

SEQ ID NO: 30 is the amino acid sequence of a wheat WRKY3 protein derived from the nucleotide sequence of SEQ ID NO: 29.

SEQ ID NO: 31 is the nucleotide sequence comprising the maize WRKY2-1 polynucleotide.

SEQ ID NO: 32 is the amino acid sequence of a maize WRKY2-1 protein derived from the nucleotide sequence of SEQ ID NO: 31.

SEQ ID NO: 33 is the nucleotide sequence comprising the maize WRKY3-2 polynucleotide.

SEQ ID NO: 34 is the amino acid sequence of a maize WRKY3-2 protein derived from the nucleotide sequence of SEQ ID NO: 33.

SEQ ID NO: 35 is the nucleotide sequence comprising the transcriptional regulatory region of a sunflower WRKY1-2 polynucleotide.

SEQ ID NO: 36 is a designed oligonucleotide based upon the adapter sequence and poly T to remove clones which have a poly A tail but no cDNA.

SEQ ID NO: 37 is the nucleotide sequence comprising the maize ZmWRKY1-1 polynucleotide.

SEQ ID NO: 38 is the amino acid sequence of the maize ZmWRKY1-1 protein derived from the nucleotide sequence of SEQ ID NO: 37.

SEQ ID NO: 39 is the nucleotide sequence comprising the maize ZmWRKY1-2 polynucleotide.

SEQ ID NO: 40 is the nucleotide sequence comprising the maize ZmWRKY2-2 polynucleotide.

SEQ ID NO: 41 is the nucleotide sequence comprising the maize ZmWRKY3-3 polynucleotide.

SEQ ID NO: 42 is the nucleotide sequence comprising the maize ZmWRKY3-4 polynucleotide.

SEQ ID NO: 43 is the nucleotide sequence comprising the maize ZmWRKY3-5 polynucleotide.

### DETAILED DESCRIPTION OF THE INVENTION

Overview

The present invention provides, among other things, compositions and methods for modulating (i.e., increasing or decreasing) the level of polynucleotides and polypeptides of the present invention in plants. In particular, the polynucleotides and polypeptides of the present invention can be expressed temporally or spatially, e.g., at developmental stages, in tissues, and/or in quantities, which are uncharacteristic of non-recombinantly engineered plants. The transcriptional regulatory region of a WRKY polynucleotide, such as the sunflower WRKY1-2 polynucleotide (SEQ ID NO: 35), can be used to drive expression of a gene of interest during pathogen infection or by chemical induction. Thus, the present invention provides utility in such exemplary applications as disease resistance.

The present invention also provides isolated nucleic acid comprising polynucleotides of sufficient length and complementarity to a gene of the present invention to use as probes or amplification primers in the detection, quantitation, or isolation of gene transcripts. For example, isolated nucleic acids of the present invention can be used as probes in detecting deficiencies in the level of mRNA in screenings for desired transgenic plants, for detecting mutations in the gene (e.g., substitutions, deletions, or additions), for monitoring upregulation of expression or changes in enzyme activity in screening assays of compounds, for detection of any number of allelic variants (polymorphisms), orthologs, or paralogs of the gene, or for site directed mutagenesis in eukaryotic cells (see, e.g., U.S. Pat. No. 5,565,350). The isolated nucleic acids of the present invention can also be used for recombinant expression of their encoded polypeptides, or for use as immunogens in the preparation and/or screening of antibodies. The isolated nucleic acids of the present invention can also be employed for use in sense or antisense suppression of one or more genes of the present invention in a host cell, tissue, or plant. Attachment of chemical agents, which bind, intercalate, cleave and/or crosslink to the isolated nucleic acids of the present invention can also be used to modulate transcription or translation. In addition, the present invention relates to finding genes and promoters that respond to WRKY domains. The full-length sequence of WRKY or a subsequence of WRKY could be used alone or fused to additional sequence to determine genes and promoter that respond to WRKY domains. The present invention also provides isolated proteins comprising a polypeptide of the present invention (e.g., preproenzyme, proenzyme, or enzymes).

The isolated nucleic acids and proteins of the present invention can be used over a broad range of plant types, particularly monocots such as the species of the family Gramineae including Sorghum (e.g. S. bicolor), Oryza, Avena, Hordeum, Secale, Triticum and Zea mays, and dicots such as Glycine. The isolated nucleic acid and proteins of the present invention can also be used in species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Pisum, Phaseolus, Lolium, and Allium.

Pathogens of the invention include, but are not limited to, viruses or viroids, bacteria, insects, fungi, and the like. Viruses include tobacco or cucumber mosaic virus, ringspot virus, necrosis virus, maize dwarf mosaic virus, etc. Specific fungal and viral pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines, Fusarium solani; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusar-atrum, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia recondita f.sp. tritici, Puccinia striiformis, Pyrenophora triticirepentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophominaphaseolina, Erysiphe cichoracearum, Rhizopus oryzae, Rhizopus arrhizus, Rhizopus stolonifer, Puccinia helianthi, Verticillium dahliae, Erwinia carotovorum p.v. Carotovora, Cephalosporium acremonium, Phytophthora cryptogea, Albugo tragopogonis; Maize: Fusarium moniliforme var. subglutinans, Erwinia stewartii, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillusflavus, Bipolaris maydis O,T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatie-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganese subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi p.v. Zea, Erwinia corotovora, Cornstunt spiroplasma, Diplodia macrospora, Sclerophthora macrospora, Peronosclerospora sorghi, Peronosclerospora philippinesis, Peronosclerospora maydis, Peronosclerospora sacchari, Spacelotheca reiliana, Physopella zea, Cephalosporium maydis, Caphalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, Colletotrichum graminicola (Glomerella graminicola), Cercospora sorghi, Gloeocercospora sorghi, Ascochyta sorghina, Pseudomonas syringae p.v. syringae, Xanthomonas campestris p.v. holcicola Pseudomonas andropogonis, Puccinia purpurea, Macrophomina phaseolina, Perconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc.

Assays that measure antipathogenic activity are commonly known in the art, as are methods to quantify disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

Plasmids containing the polynucleotide sequences of the invention were deposited with American Type Culture Collection (ATCC), Manassas, Va., and assigned the following Patent Deposit Designation numbers: for maize ZmWRKY3-1 the designation is PTA-1590; for SWRKY1-1 the designation is PTA-1510, for SWRKY1-2 the designation is PTA-1504, for SWRKY1-3 the designation is PTA-1511, for SWRKY1-4 the designation is PTA-1509, and for the 5′ regulatory region of WRKY1-2 the designation is PTA-1505. These deposits will be maintained under the terms of the Budapest Treaty on the International Recognition of the Deposit of Microorganisms for the Purposes of Patent Procedure. These deposits were made merely as a convenience for those of skill in the art and are not an admission that a deposit is required under 35 U.S.C. § 112.

Definitions

Units, prefixes, and symbols may be denoted in their SI accepted form. Unless otherwise indicated, nucleic acids are written left to right in 5′ to 3′ orientation, amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D H Persing et al., Ed., American Society for Microbiology, Washington, D.C. (1993). The product of amplification is termed an amplicon.

As used herein, “antisense orientation” includes reference to a duplex polynucleotide sequence, which is operably linked to a promoter in an orientation where the antisense strand is transcribed. The antisense strand is sufficiently complementary to an endogenous transcription product such that translation of the endogenous transcription product is often inhibited.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fingal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). Thus, the maize preferred codon for a particular amino acid might be derived from known gene sequences from maize. Maize codon usage for 28 genes from maize plants is listed in Table 4 of Murray et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species, or, if from the same species, is substantially modified from its original form by deliberate human intervention.

By “host cell” is meant a cell, which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids, which are “isolated”, as defined herein, are also referred to as “heterologous” nucleic acids.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, ajoint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants, which can be used in the methods of the invention, is generally as broad as the class of higher plants amenable to transformation techniques, including both monocotyledonous and dicotyledonous plants. Preferred plants include, but are not limited to maize, soybean, sunflower, sorghum, canola, wheat, alfalfa, cotton, rice, barley, and millet. A particularly preferred plant is maize (Zea mays).

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modification have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide”, and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquination, and they may be circular, with or without branching, generally as a result of post-translation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine containing and the methionine-less amino terminal variants of the protein of the invention.

As used herein “promoter or transcriptional regulatory region” includes reference to a region of DNA upstream from the start of transcription and involved in recognition and binding of RNA polymerase and other proteins to initiate transcription. A “plant promoter or transcriptional regulatory region” is a promoter or transcriptional regulatory region capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters who initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter, which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter, which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes a reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC =3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1× SSC at 55 to 60° C. Exemplary high stringency conditions include hybridization in 50% formamide, I M NaCl, 1% SDS at 37° C., and a wash in0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (%CG)−0.61 (% form)−500/L; where M is the molarity of monovalent cations, %CG is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides: (a) “reference sequence”, (b) “comparison windows”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Optimal alignment of sequences for comparison may be conducted by the local homology algorithm of Smith and Waterman. Adv. Appl. Math. 2: 482 (1981); by the homology alignment algorithm of Needleman and Wunsch, J. Mol Biol 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

GAP uses the algorithm of Needleman and Wunsch (J Mol Biol 48: 443-453 (1970)) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the over the length of the gap times the gap extension penalty. Default gap creation penalty values and gap extension penalty values in Version 10 of the Wisconsin Genetics Software Package are 8 and 2, respectively, for protein sequences. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50, 60, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff, Proc Natl Acad Sci USA 89:10915). Unless otherwise stated, sequence identity/similarity values provided herein refer to the value obtained using the GAP version 10 of Wisconsin Genetic Software Package using default parameters.

Comparisons of polynucleotide sequences that are of substantially different lengths can be determined by a combination of percent identity between the two sequences times the ratio of the coding region. In other words, Relation=% Identity×Ratio of the coding region. For example, if a first polynucleotide is 100% identical at the nucleotide level, but only represents 30% of the coding region of the second polynucleotide, then it is expressed as 30% related.

(c) As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Califormia, USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Nucleic Acids

The present invention provides, among other things, isolated nucleic acids of RNA, DNA, and analogs and/or chimeras thereof, comprising a polynucleotide of the present invention.

A polynucleotide of the present invention is inclusive of:

(a) a polynucleotide encoding a polypeptide of SEQ ID NOS: 2, 10, 12, 14, 16, 18, 20, 22, 24, 26, 28, 30, 32, 34, and 38, including exemplary polynucleotides of SEQ ID NOS: 1, 9, 11, 13, 15, 17, 19, 21, 23, 25, 27, 29, 31, 33, 37, 39, 40, 41, 42, and 43;

(b) a polynucleotide which is the product of amplification from a Zea mays nucleic acid library using primer pairs which selectively hybridize under stringent conditions to loci within a polynucleotide selected from the group consisting of SEQ ID NOS: 1, 9, 11, 13, 15, 17, 19, 21, 23, 25, 27, 29, 31, 33, 37, 39, 40, 41, 42, and 43, wherein the polynucleotide has substantial sequence identity to a polynucleotide selected from the group consisting of SEQ ID NOS: 1, 9, 11, 13, 15, 17, 19, 21, 23, 25, 27, 29, 31, 33, 37, 39, 40, 41, 42, and 43;

(c) a polynucleotide which selectively hybridizes to a polynucleotide of (a) or (b);

(d) a polynucleotide having a specified sequence identity with polynucleotides of (a), (b), or (c);

(e) complementary sequences of polynucleotides of (a), (b), (c), r (d); and

(f) a polynucleotide comprising at least a specific number of contiguous nucleotides from a polynucleotide of (a), (b), (c), (d), or (e).

A. Polynucleotides Encoding a Polypeptide of the Present Invention

The present invention provides isolated nucleic acids comprising a polynucleotide of the present invention, wherein the polynucleotide encodes a polypeptide of the present invention. Accordingly, the present invention includes polynucleotides of SEQ ID NOS: 1, 9, 11, 13, 15, 17, 19, 21, 23, 25, 27, 29, 31, 33, 37, 39, 40, 41, 42, and 43, and silent variations of polynucleotides encoding a polypeptide of SEQ ID NOS: 2, 10, 12, 14, 16, 18, 20, 22, 24, 26, 28, 30, 32, 34, and 38. The present invention further provides isolated nucleic acids comprising polynucleotides encoding conservatively modified variants of a polypeptide of SEQ ID NOS: 2, 10, 12, 14, 16, 18, 20, 22, 24, 26, 28, 30, 32, 34, and 38. Conservatively modified variants can be used to generate or select antibodies immunoreactive to the non-variant polypeptide. Additionally, the present invention further provides isolated nucleic acids comprising polynucleotides encoding one or more allelic (polymorphic) variants of polypeptides/polynucleotides. Polymorphic variants are frequently used to follow segregation of chromosomal regions in, for example, marker assisted selection methods for crop improvement.

B. Polynucleotides Amplified from a Plant Nucleic Acid Library

The present invention provides an isolated nucleic acid comprising a polynucleotide of the present invention, wherein the polynucleotides are amplified, under nucleic acid amplification conditions, from a plant nucleic acid library. Nucleic acid amplification conditions for each of the variety of amplification methods are well known to those of ordinary skill in the art. The plant nucleic acid library can be constructed from a monocot such as a cereal crop. Exemplary cereals include corn, sorghum, alfalfa, canola, wheat, or rice. The plant nucleic acid library can also be constructed from a dicot such as soybean. Zea mays lines B73, PHRE1, A632, BMS-P2#10, W23, and Mol7 are known and publicly available. Other publicly known and available maize lines can be obtained from the Maize Genetics Cooperation (Urbana, Ill.). Wheat lines are available from the Wheat Genetics Resource Center (Manhattan, Kans.).

The nucleic acid library may be a cDNA library, a genomic library, or a library generally constructed from nuclear transcripts at any stage of intron processing. cDNA libraries can be normalized to increase the representation of relatively rare cDNAs. In optional embodiments, the cDNA library is constructed using an enriched full-length cDNA synthesis method. Examples of such methods include Oligo-Capping (Maruyama, K. and Sugano, S. Gene 138: 171-174, 1994), Biotinylated CAP Trapper (Carninci, et al. Genomics 37: 327-336, 1996), and CAP Retention Procedure (Edery, E., Chu, L. L., et al. Molecular and Cellular Biology 15: 3363-3371, 1995). Rapidly growing tissues or rapidly dividing cells are preferred for use as a mRNA source for construction of a cDNA library. Growth stages of corn is described in “How a Corn Plant Develops,” Special Report No. 48, Iowa State University of Science and Technology Cooperative Extension Service, Ames, Iowa, Reprinted February 1993.

A polynucleotide of this embodiment (or subsequences thereof) can be obtained, for example, by using amplification primers which are selectively hybridized and primer extended, under nucleic acid amplification conditions, to at least two sites within a polynucleotide of the present invention, or to two sites within the nucleic acid which flank and comprise a polynucleotide of the present invention, or to a site within a polynucleotide of the present invention and a site within the nucleic acid which comprises it. Methods for obtaining 5′ and/or 3′ ends of a vector insert are well known in the art. See, e.g., RACE (Rapid Amplification of Complementary Ends) as described in Frohman, M. A., in PCR Protocols: A Guide to Methods and Applications, M. A. Innis, D. H. Gelfand, J. J. Sninsky, T. J. White, Eds. (Academic Press, Inc., San Diego), pp. 28-38 (1990)); see also, U.S. Pat. No. 5,470,722, and Current Protocols in Molecular Biology, Unit 15.6, Ausubel, et al., Eds., Greene Publishing and Wiley-Interscience, New York (1995); Frohman and Martin, Techniques 1:165 (1989).

Preferably, the primers are complementary to a subsequence of the target nucleic acid which they amplify but may have a sequence identity ranging from about 85% to 99% relative to the polynucleotide sequence which they are designed to anneal to. As those skilled in the art will appreciate, the sites to which the primer pairs will selectively hybridize are chosen such that a single contiguous nucleic acid can be formed under the desired nucleic acid amplification conditions. The primer length in nucleotides is selected from the group of integers consisting of from at least 15 to 50. Thus, the primers can be at least 15, 18, 20, 25, 30, 40, or 50 nucleotides in length. Those of skill will recognize that a lengthened primer sequence can be employed to increase specificity of binding (i.e., annealing) to a target sequence. A non-annealing sequence at the 5′ end of a primer (a “tail”) can be added, for example, to introduce a cloning site at the terminal ends of the amplicon.

The amplification products can be translated using expression systems well known to those of skill in the art. The resulting translation products can be confirmed as polypeptides of the present invention by, for example, assaying for the appropriate catalytic activity (e.g., specific activity and/or substrate specificity), or verifying the presence of one or more linear epitopes, which are specific to a polypeptide of the present invention. Methods for protein synthesis from PCR derived templates are known in the art and available commercially. See, e.g., Amersham Life Sciences, Inc, Catalog '97, p.354.

C. Polynucleotides Which Selectively Hybridize to a Polynucleotide of (A) or (B)

The present invention provides isolated nucleic acids comprising polynucleotides of the present invention, wherein the polynucleotides selectively hybridize, under selective hybridization conditions, to a polynucleotide of section (A) or (B) as discussed above. Thus, the polynucleotides of this embodiment can be used for isolating, detecting, and/or quantifying nucleic acids comprising the polynucleotides of (A) or (B). For example, polynucleotides of the present invention can be used to identify, isolate, or amplify partial or full-length clones in a deposited library. In some embodiments, the polynucleotides are genomic or cDNA sequences isolated or otherwise complementary to a cDNA from a dicot or monocot nucleic acid library. Exemplary species of monocots and dicots include, but are not limited to: maize, canola, soybean, cotton, wheat, sorghum, sunflower, alfalfa, oats, sugar cane, millet, barley, and rice. The cDNA library comprises at least 50% to 95% full-length sequences (for example, at least 50%, 60%, 70%, 80%, 90%, or 95% full-length sequences). The cDNA libraries can be normalized to increase the representation of rare sequences. See, e.g., U.S. Pat. No. 5,482,845. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% to 80% sequence identity and can be employed to identify orthologous or paralogous sequences.

D. Polynucleotides Having a Specific Sequence Identify with the Polynucleotides of (A), (B) or (C)

The present invention provides isolated nucleic acids comprising polynucleotides of the present invention, wherein the polynucleotides have a specified identity at the nucleotide level to a polynucleotide as disclosed above in sections (A), (B), or (C), above. The percentage of identity to a reference sequence is at least 60% and, rounded upwards to the nearest integer, can be expressed as an integer selected from the group of integers consisting of from 60 to 99. Thus, for example, the percentage of identity to a reference sequence can be at least 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Optionally, the polynucleotides of this embodiment will encode a polypeptide that will share an epitope with a polypeptide encoded by the polynucleotides of section (A), (B), or (C). Thus, these polynucleotides encode a first polypeptide, which elicits production of antisera comprising which are specifically reactive to a second polypeptide encoded by a polynucleotide of (A), (B), or (C). However, the first polypeptide does not bind to antisera raised against itself when the antisera have been fully immunosorbed with the first polypeptide. Hence, the polynucleotides of this embodiment can be used to generate antibodies for use in, for example, the screening of expression libraries for nucleic acids comprising polynucleotides of (A), (B), or (C), or for purification of, or in immunoassays for, polypeptides encoded by the polynucleotides of (A), (B), or (C). The polynucleotides of this embodiment embrace nucleic acid sequences, which can be employed for selective hybridization to a polynucleotide encoding a polypeptide of the present invention.

Screening polypeptides for specific binding to antisera can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 15 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT patent publication Nos. 91/17271, 91/18980, 91/19818, and 93/08278. Other systems for generating libraries of peptides have aspects of both in vitro chemical synthesis and recombinant methods. See PCT Patent publication Nos. 92/05258, 92/14843, and 96/19256. See also, U.S. Pat. Nos. 5,658,754; and 5,643,768. Peptide display libraries, vectors, and screening kits are commercially available from such suppliers as Invitrogen (Carlsbad, Calif.).

E. Polynucleotides Complementary to the Polynucleotides of (A)-(D).

The present invention provides isolated nucleic acids comprising polynucleotides complementary to the polynucleotides of paragraphs A-D, above. As those of skill in the art will recognize, complementary sequences base-pair throughout the entirety of their length with the polynucleotides of sections (A)-(D) (i.e., have 100% sequence identity over their entire length.) Complementary bases associate through hydrogen bonding in double stranded nucleic acids. For example, the following base pairs are complementary: guanine and cytosine; adenine and thymine; and adenine and uracil.

F. Polynucleotides That are Subsequences of the Polynucleotides of (A)-(E)

The present invention provides isolated nucleic acids comprising polynucleotides that comprise at least 15 contiguous bases from the polynucleotides of section (A) through (E) as discussed above. The length of the polynucleotide is given as an integer selected from the group consisting of from at least 15 to the length of the nucleic acid sequence from which the polynucleotide is a subsequence of. Thus, for example, polynucleotides of the present invention are inclusive of polynucleotides comprising at least 15, 20, 25, 30, 40, 50, 60, 75, or 100 contiguous nucleotides in length from the polynucleotides of (A)-(E). Optionally, the number of such subsequences encoded by a polynucleotide of the instant embodiment can be any integer selected from the group consisting of from 1 to 20, such as 2, 3, 4, or 5. The subsequences can be separated by any integer of nucleotides from 1 to the number of nucleotides in the sequence such as at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100, 110, 120, 130, 140, 150, 160, 170, 180, 190, 200, 210, 220, 230, 240, 250, 260, 270, 280, 290, 300, 350, 400, 450, 500, 550, 600, 650, 700, 750, 800, 850, 900, 950, or 1000.

The subsequences of the present invention can comprise structural characteristics of the sequence from which it is derived. Alternatively, the subsequences can lack certain structural characteristics of the larger sequence from which it is derived such as poly (A) tail. Optionally, a subsequence from a polynucleotide encoding a polypeptide having at least one linear epitope in common with a prototype polypeptide sequence as provided in (a), above, may encode an epitope in common with the prototype sequence. Alternatively, the subsequence may not encode an epitope in common with the prototype sequence but can be used to isolate the larger sequence by, for example, nucleic acid hybridization with the sequence from which it's derived. Subsequences can be used to modulate or detect gene expression by introducing into the subsequence compounds, which bind, intercalate, cleave and/or crosslink to nucleic acids. Exemplary compounds include acridine, psoralen, phenanthroline, naphthoquinone, daunomycin, or chloroethylaminoaryl conjugates. In addition, by virtue of the fact that WRKY polynucleotides contain DNA binding regions, such as the TGAC-containing W box, subsequences of a WRKY polynucleotide could be used to test the binding of target DNA or to identify genes or promoters that respond to the WRKY domains.

Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot. In preferred embodiments the monocot is Zea mays.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. A polynucleotide of the present invention can be attached to a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb, and frequently less than 10 kb. Use of cloning vectors, expression vectors, adapters, and linkers is well known and extensively described in the art. For a description of various nucleic acids see, for example, Stratagene Cloning Systems, Catalogs 1999 (La Jolla, Calif.); and, Amersham Life Sciences, Inc, Catalog '99 (Arlington Heights, Ill.).

A. Recombinant Methods for Constructing Nucleic Acids

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. Isolation of RNA and construction of cDNA and genomic libraries is well known to those of ordinary skill in the art. See, e.g., Plant Molecular Biology: A Laboratory Manual, Clark, Ed., Springer-Verlag, Berlin (1997); and, Current Protocols in Molecular Biology, Ausubel, et al., Eds., Greene Publishing and Wiley-Interscience, New York (1995).

A1. Full-length Enriched cDNA Libraries

A number of cDNA synthesis protocols have been described which provide enriched full-length cDNA libraries. Enriched full-length cDNA libraries are constructed to comprise at least 60%, and more preferably at least 70%, 80%, 90% or 95% full-length inserts amongst clones containing inserts. The length of insert in such libraries can be at least 2, 3, 4, 5, 6, 7, 8, 9, 10 or more kilobase pairs. Vectors to accommodate inserts of these sizes are known in the art and available commercially. See, e.g., Stratagene's lambda ZAP Express (cDNA cloning vector with 0 to 12 kb cloning capacity). An exemplary method of constructing a greater than 95% pure full-length cDNA library is described by Carninci et al., Genomics, 37:327-336 (1996). Other methods for producing full-length libraries are known in the art. See, e.g., Edery et al., Mol. Cell Biol., 15(6):3363-3371 (1995); and, PCT Application WO 96/34981.

A2 Normalized or Subtracted cDNA Libraries

A non-normalized cDNA library represents the mRNA population of the tissue it was made from. Since unique clones are out-numbered by clones derived from highly expressed genes their isolation can be laborious. Normalization of a cDNA library is the process of creating a library in which each clone is more equally represented. Construction of normalized libraries is described in Ko, Nucl Acids Res, 18(19):5705-5711 (1990); Patanjali et al., Proc. Natl. Acad. U.S.A., 88:1943-1947 (1991); U.S. Pat. Nos. 5,482,685, 5,482,845, and 5,637,685. In an exemplary method described by Soares et al., normalization resulted in reduction of the abundance of clones from a range of four orders of magnitude to a narrow range of only 1 order of magnitude. Proc. Natl. Acad. Sci. USA, 91:9228-9232 (1994).

Subtracted cDNA libraries are another means to increase the proportion of less abundant cDNA species. In this procedure, cDNA prepared from one pool of mRNA is depleted of sequences present in a second pool of mRNA by hybridization. The cDNA:mRNA hybrids are removed and the remaining un-hybridized cDNA pool is enriched for sequences unique to that pool. See, Foote et al. in, Plant Molecular Biology: A Laboratory Manual, Clark, Ed., Springer-Verlag, Berlin (1997); Kho and Zarbl, Technique, 3(2):58-63 (1991); Sive and St. John, Nucl. Acids Res., 16(22):10937 (1988); Current Protocols in Molecular Biology, Ausubel, et al., Eds., Greene Publishing and Wiley-Interscience, New York (1995); and, Swaroop et al., Nucl. Acids Res., 19(8):1954 (1991). cDNA subtraction kits are commercially available. See, e.g., PCR-Select (Clontech, Palo Alto, Calif.).

To construct genomic libraries, large segments of genomic DNA are generated by fragmentation, e.g. using restriction endonucleases, and are ligated with vector DNA to form concatemers that can be packaged into the appropriate vector. Methodologies to accomplish these ends and sequencing methods to verify the sequence of nucleic acids are well known in the art. Examples of appropriate molecular biological techniques and instructions sufficient to direct persons of skill through many construction, cloning, and screening methodologies are found in Sambrook, et al., Molecular Cloning: A Laboratory Manual, 2nd Ed., Cold Spring Harbor Laboratory Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel, et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a polynucleotide of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent.

The nucleic acids of interest can also be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. The T4 gene 32 protein (Boehringer Mannheim) can be used to improve yield of long PCR products.

PCR-based screening methods have been described. Wilfinger et al. describe a PCR-based method in which the longest cDNA is identified in the first step so that incomplete clones can be eliminated from study. BioTechniques, 22(3): 481-486 (1997). Such methods are particularly effective in combination with a full-length cDNA construction methodology, above.

B. Synthetic Methods for Constructing Nucleic Acids

The isolated nucleic acids of the present invention can also be prepared by direct chemical synthesis by methods such as the phosphotriester method of Narang et al., Meth. Enzymol. 68: 90-99 (1979); the phosphodiester method of Brown et al., Meth. Enzymol. 68: 109-151 (1979); the diethylphosphoramidite method of Beaucage et al., Tetra. Lett. 22: 1859-1862 (1981); the solid phase phosphoramidite triester method described by Beaucage and Caruthers, Tetra. Letts. 22(20): 1859-1862 (1981), e.g., using an automated synthesizer, e.g., as described in Needham-VanDevanter et al., Nucleic Acids Res., 12: 6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is best employed for sequences of about 100 bases or less, longer sequences may be obtained by the ligation of shorter sequences.

Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a full length polypeptide of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plan expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used in the practice of the invention. A plant promoter fragment can be employed which will direct expression of a polynucleotide of the present invention in all tissues of a regenerated plant. Such promoters are referred to herein as “constitutive” promoters and are active under most environmental conditions and stated of development or cell differentiation. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter (Christensen, et al. Plant Mol Biol 18, 675-689 (1992); Bruce, et al., Proc Natl Acad Sci USA 86, 9692-9696 (1989)), the Smas promoter, the cinnamyl alcohol dehydrogenase promoter (U.S. Pat. No, 5,683,439), the Nos promoter, the pEmu promoter, the rubisco promoter, the GRP 1-8 promoter, the maize constitutive promoters described in PCT Publication No. WO 99/43797 which include the histone H2B, metallothionein, alpha-tubulin 3, elongation factor efla, ribosomal protein rps8, chlorophyll a/b binding protein, and glyceraldehyde-3-phosphate dehydrogenase promoters, and other transcription initiation regions from various plant genes known to those of skill. The preferred promoter is a pathogen-inducible promoter such as the Sclerotinia-inducible promoters PR5-2 and BAP, which can be found in co-pending U.S. application number 09/185,292, filed Oct. 10, 2000. Another preferred inducible promoter is a promoter designed with the estrogen response element (ERE) (Klein-Hitpass, et al., Nuc. Acids Res. 16:647-63 (1988)). For example, four repeats of the ERE element are fused upstream of the Adhl minimal promoter, which is fused upstream of the Adhl intron.

Where low level expression is desired, weak promoters will be used. It is recognized that weak inducible promoters may be used. Additionally, either a weak constitutive or a weak tissue specific promoter may be used. Generally, by a “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompass promoters that are expresses in only a few cells and not in others to give a total low level of expression. Such weak constitutive promoters include, for example, the core promoter of the Rsyn7 (WO 97/44756), the core 35S CaMV promoter, and the like. Where a promoter is expressed at unacceptably high levels, portions of the promoter sequence can be deleted or modified to decrease expression levels. Additionally, to obtain a varied series in the level of expression, one can also make a set of transgenic plants containing the polynucleotides of the present invention with a strong constitutive promoter, and then rank the transgenic plants according to the observed level of expression. The transgenic plants will show a variety in performance, from high expression to low expression. Factors such as chromosomal position effect, cosuppression, and the like will affect the expression of the polynucleotide.

Alternatively, the plant promoter can direct expression of a polynucleotide of the present invention under environmental control. Such promoters are referred to here as “inducible” promoters. Environmental conditions that may effect transcription by inducible promoters include pathogen attack, anaerobic conditions, or the presence of light. Examples of inducible promoters are the Adhl promoter, which is inducible by hypoxia or cold stress, the Hsp7O promoter, which is inducible by heat stress, and the PPDK promoter, which is inducible by light. Examples of pathogen-inducible promoters include those from proteins, which are induced following infection by a pathogen; e.g., PR proteins, SAR proteins, beta-a,3-glucanase, chitinase, etc. See, for example, Redolfi, et al., Meth J. Plant Pathol. 89:245-254 (1983); Uknes et al., The Plant Cell 4:645-656 (1992); Van Loon, Plant Mol. Virol. 4:111-116 (1985); and PCT Publication No. WO 99/43819.

Of interest are promoters that are expresses locally at or near the site of pathogen infection. See, for example, Marineau, et al., Plant Mol Biol 9:335-342 (1987); Matton, et al., Molecular Plant-Microbe Interactions 2:325-342 (1987); Somssich et al., Proc Natl AcadSci USA 83:2427-2430 (1986); Somssich et al., Mole Gen Genetics 2:93-98 (1988); Yang, Proc Natl Acad Sci USA 93:14972-14977. See also, Chen, et al., Plant J 10:955-966 (1996); Zhang and Sing, Proc Natl Acad Sci USA 91:2507-2511 (1994); Warner, et al., Plant J 3:191-201 (1993), and Siebertz, et al., Plant Cell 1:961-968 (1989), all of which are herein incorporated by reference. Of particular interest is the inducible promoter for the maize PRms gene, whose expression is induced by the pathogen Fusarium moniliforme (see, for example, Cordero, et al., Physiol Molec Plant Path 41:189-200 (1992) and is herein incorporated by reference.

Additionally, as pathogens find entry into plants through wounds or insect damage, a wound inducible promoter may be used in the constructs of the invention. Such wound inducible promoters include potato proteinase inhibitor (pin II) gene (Ryan, Annu Rev Phytopath 28:425-449 (1990); Duan, et al., Nat Biotech 14:494-498 (1996)); wun1 and wun 2, U.S. Pat. No. 5,428,148; win1 and win2 (Stanford et al., Mol Gen Genet 215:200-208 (1989)); systemin (McGurl, et al., Science 225:1570-1573 (1992)); WIP1 (Rohmeier, et al., Plant Mol Biol 22:783-792 (1993); Eckelkamp, et al., FEB Letters 323:73-76 (1993)); MPI gene (Corderok, et al., The Plant J 6(2):141-150(1994)); and the like, herein incorporated by reference.

Examples of promoters under developmental control include promoters that initiate transcription only, or preferentially, in certain tissues, such as leaves, roots, fruit, seeds, or flowers. Exemplary promoters include the anther specific promoter 5126 (U.S. Pat. Nos. 5,689,049 and 5,689,051), glob-1 promoter, and gamma-zein promoter. An exemplary promoter for leaf- and stalk-preferred expression is MS8-15 (WO 98/00533). Examples of seed-preferred promoters included, but are not limited to, 27 kD gamma zein promoter and waxy promoter (Boronat, et al., Plant Sci, 47:95-102 (1986); Reina, et al., Nucleic Acids Res 18(21):6426 (1990); and Kloesgen, et al., Mol Gen Genet 203:237-244 (1986)). Promoters that express in the embryo, pericarp, and endosperm are disclosed in PCT Publication WO 00/11177, published on Mar. 2, 2000, and PCT Publication WO 00/12733, published on Mar. 9, 2000, both of which are hereby incorporated by reference. The operation of a promoter may also vary depending on its location in the genome. Thus, a developmentally regulated promoter may become fully or partially constitutive in certain locations. A developmentally regulated promoter can also be modified, if necessary, for weak expression.

Both heterologous and non-heterologous (i.e. endogenous) promoters can be employed to direct expression of the nucleic acids of the present invention. These promoters can also be used, for example, in recombinant expression cassettes to drive expression of antisense nucleic acids to reduce, increase, or alter concentration and/or composition of the proteins of the present invention in a desired tissue. Thus, in some embodiments, the nucleic acid construct will comprise a promoter functional in a plant cell, such as in Zea Mays, operably linked to a polynucleotide of the present invention. Promoters useful in these embodiments include the endogenous promoters driving expression of a polypeptide of the present invention.

In some embodiments, isolated nucleic acids which serve as a promoter or enhancer elements can be introduced in the appropriate position (generally upstream) of a non-heterologous form of a polynucleotide of the present invention so as to up or down regulate expression of a polynucleotide of the present invention. For example, endogenous promoters can be altered in vivo by mutation, deletion, and/or substitution (see, Kmiec, U.S. Pat. No. 5,565,350; Zarling et al., PCT/US93?03868), or isolated promoters can be introduced into a plant cell in the proper orientation and distance from a gene of the present invention so as to control the expression of the gene. Gene expression can be modulated under conditions suitable for plant growth so as to alter the total concentration and/or alter the composition of the polypeptides of the present invention in plant cell. Thus, the present invention provides compositions, and methods for making, heterologous promoters and/or enhancers operably linked to a native, endogenous (i.e., non-heterologous) form of a polynucleotide of the present invention.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or less preferably from any other eukaryotic gene.

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold, Buchman and Berg, Mol. Cell Biol. 8: 4395-4405 (1988); Callis et al., Genes Dev. 1: 1183-1200 (1987). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS gene encodes resistance to the herbicide chlorsulfuron.

Typical vectors useful for expression of genes in higher plants are well known in the art and include vectors derived from the tumor-induced (Ti) plasmid of Agrobacterium tumefaciens described by Rogers et al., Meth. In Enzymol., 153:253-277 (1987). These vectors are plant integrating vectors in that upon transformation, the vectors integrate a portion of vector DNA into the genome of the host plant. Exemplary A. tumefaciens vectors useful herein are plasmids pKYLX6 and pKYLX7 of Schardl et al., Gene, 61:1-11(1987) and Berger et al., Proc. Natl. Acad. Sci. U.S.A., 86:8402-8406 (1989). Another useful vector herein is plasmid pBI101.2 that is available from Clontech Laboratories, Inc. (Palo Alto, Calif.).

A polynucleotide of the present invention can be expressed in either sense or anti-sense orientation as desired. It will be appreciated that control of gene expression in either sense or anti-sense orientation can have a direct impact on the observable plant characteristics. Antisense technology can be conveniently used to inhibit gene expression in plants. To accomplish this, a nucleic acid segment from the desired gene is cloned and operably linked to a promoter such that the anti-sense strand of RNA will be transcribed. The construct is then transformed into plants and the antisense strand of RNA is produced. In plant cells, it has been shown that antisense RNA inhibits gene expression by preventing the accumulation of mRNA which encodes the enzyme of interest, see, e.g., Sheehy et al., Proc. Nat'l. Acad. Sci (USA) 85:8805-8809 (1988); and Hiatt et al., U.S. Pat. No. 4,801,340.

Another method of suppression is sense suppression. Introduction of nucleic acid configured in the sense orientation has been shown to be an effective means by which to block the transcription of target genes. For an example of the use of this method to modulate expression of endogenous genes see, Napoli et al., The Plant Cell 2:279-289 (1990) and U.S. Pat. No. 5,034,323.

Catalytic RNA molecules or ribozymes can also be used to inhibit expression of plant genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., Nucleic Acids Res (1986) 14:4065-4076, describe covalent bonding of a single-stranded DNA fragment with alkylating derivatives of nucleotides complementary to target sequences. A report of similar work by the same group is that by Knorre, D. G., et al., Biochimie (1985) 67:785-789. Iverson and Dervan also showed sequence-specific cleavage of single-stranded DNA meditated by incorporation of a modified nucleotide which was capable of activating cleavage (J Am Chem Soc (1987) 109:1241-1243). Meyer, R. B. et al., J Am Chem Soc (1989) 111:8517-8519, effect covalent crosslinking to a target nucleotide using an alkylating agent complementary to the single-stranded target nucleotide sequence. A photoactivated crosslinking to single-stranded oligonucleotides meditated by psoralen was disclosed by Lee, B. L., et al., Biochemistry (1988) 27:3197-3203. Use of crosslinking in triple-helix forming probes was also disclosed by Home et al., J. Am Chem Soc (1990) 112:2435-2437. Use of N4, N4-ethanocytosine as an alkylating agent to crosslink to single-stranded oligonucleotides has also been described by Webb and Matteucci, J Am Chem Soc (1986) 108:2764-2765; Nucleic Acids Res (1986) 14:7661-7674; Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681,941.

Proteins

The isolated proteins of the present invention comprise a polypeptide having at least 10 amino acids encoded by any one of the polynucleotides of the present invention as discussed more fully, above, or polypeptides which are conservatively modified variants thereof. The proteins of the present invention or variants thereof can comprise any number of contiguous amino acid residues from a polypeptide of the present invention, wherein that number is selected from the group of integers consisting of from 10 to the number of residues in a full-length polypeptide of the present invention. Optionally, this subsequence of contiguous amino acids is at least 15, 20, 25, 30, 35, or 40 amino acids in length, often at least 50, 60, 70, 80, or 90 amino acids in length. Further, the number of such subsequences can be any integer selected from the group consisting of from 1 to 20, such as 2, 3, 4, or 5.

As those of skill will appreciate, the present invention includes catalytically active polypeptides of the present invention (i.e., enzymes). Catalytically active polypeptides have a specific activity of at least 20%, 30%, or 40%, and preferably at least 50%, 60%, or 70%, and most preferably at least 80%, 90%, or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, or 50%, that of the native (non-synthetic), endogenous polypeptide; and more preferably at least 60%, 70%, 80%, or 90%. Methods of assaying and quantifying measures of enzymatic activity and substrate specificity (k_{cat}/Kₘ), are well known to those of skill in the art.

Generally, the proteins of the present invention will, when presented as an immunogen, elicit production of an antibody specifically reactive to a polypeptide of the present invention. Further, the proteins of the present invention will not bind to antisera raised against a polypeptide of the present invention, which has been fully immunosorbed with the same polypeptide. Immunoassays for determining binding are well known to those of skill in the art. A preferred immunoassay is a competitive immunoassay as discussed, infra. Thus, the proteins of the present invention can be employed as immunogens for constructing antibodies immunoreactive to a protein of the present invention for such exemplary utilities as immunoassays or protein purification techniques.

Expression of Proteins in Host Cells

Using the nucleic acids of the present invention, one may express a protein of the present invention in a recombinantly engineered cell such as bacteria, yeast, insect, mammalian, or preferably plant cells. The cells produce the protein in a non-natural condition. (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or regulatable), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator. One of skill would recognize that modifications could be made to a protein of the present invention without diminishing its biological activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located purification sequences. Restriction sites or termination codons can also be introduced.

A. Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al., Nature 198:1056 (1977)), the tryptophan (trp) promoter system (Goeddel et al., Nucleic Acids Res. 8:4057 (1980)) and the lambda derived P L promoter and N-gene ribosome binding site (Shimatake et al., Nature 292:128(1981)). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al., Gene 22: 229-235 (1983); Mosbach, et al., Nature 302:543-545 (1983)).

B. Expression in Eukaryotes

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, F., et al., Methods in Yeast Genetics, Cold Spring Harbor Laboratory (1982) is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysine the cells and applying standard protein isolation techniques to the lists. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect, or plant origin. Illustrative cell cultures useful for the production of the peptides are mammalian cells. Mammalian cell systems often will be in the form of minelayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al., Immunol. Rev. 89:49 (1986)), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider, J. Embryol. Exp. Morphol. 27:353-365 (1987).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP 1 intron from SV40 (Sprague, et al., J. Virol. 45:773-781 (1983)). Additionally, gene sequences to control replication in the host cell may be incorporated into the vector such as those found in bovine papilloma virus type-vectors. Saveria-Campo, M., Bovine Papilloma Virus DNA a Eukaryotic Cloning Vector in DNA Cloning Vol. II a Practical Approach, D. M. Glover, Ed., IRL Press, Arlington, Virginia pp. 213-238(1985).

Transfection/Transformation of Cells

The method of transfortnation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method, which provides for effective transformation/transfection may be employed.

A. Plant Transformation

The genes of the present invention can be used to transform any plant. In this manner, genetically modified plants, plant cells, plant tissue, seed, and the like can be obtained. Transformation protocols may vary depending on the type of plant cell, i.e. monocot or dicot, targeted for transformation. Suitable methods of transforming plant cells include microinjection (Crossway et al., (1986) BioTechniques 4:320-334), electroporation (Riggs et al., (1986) Proc. Natl. Acad. Sci. USA 83:5602-5606, Agrobacterium mediated transformation (Hinchee et al., (1988) Biotechnology 6:915-921), direct gene transfer (Paszkowski et al., (1984) EMBO J. 3:2717-2722), and ballistic particle acceleration (see, for example, Sanford et al., U.S. Pat. No. 4,945,050; Tomes et al., “Direct DNA Transfer into Intact Plant Cells via Microprojectile Bombardment” In Gamborg and Phillips (Eds.) Plant Cell, Tissue and Organ Culture: Fundamental Methods, Springer-Verlag, Berlin (1995); and McCabe et al., (1988) Biotechnology 6:923-926). Also see, Weissinger et al., (1988) Annual Rev. Genet. 22:421-477; Sanford et al., (1987) Particulate Science and Technology 5:27-37 (onion); Christou et al., (1988) Plant Physiol. 87:671-674 (soybean); McCabe et al., (1988) Bio/Technology 6:923-926 (soybean); Datta et al., (1990) Biotechnology 8:736-740 (rice); Klein et al., (1988) Proc. Natl. Acad. Sci. USA 85:4305-4309 (maize); Klein et al., (1988) Biotechnology 6:559-563 (maize); Tomes et al., “Direct DNA Transfer into Intact Plant Cells via Microprojectile Bombardment” in Gamborg and Phillips (Eds.) Plant Cell, Tissue and Organ Culture: Fundamental Methods, Springer-Verlag, Berlin (1995) (maize); Klein et al., (1988) Plant Physiol. 91:440-444 (maize) Fromm et al., (1990) Biotechnology 8:833-839 (maize); Hooydaas-Van Slogteren & Hooykaas (1984) Nature (London) 311:763-764; Bytebier et al., (1987) Proc. Natl. Acad. Sci. USA 84:5345-5349 (Liliaceae); De Wet et al., (1985) In The Experimental Manipulation of Ovule Tissues ed. G. P. Chapman et al., pp. 197-209. Longman, N.Y. (pollen); Kaeppler et al., (1990) Plant Cell Reports 9:415-418; and Kaeppler et al., (1992) Theor. Appl. Genet. 84:560-566 (whisker-meditated transformation); D-'Halluin et al., (1992) Plant Cell 4:1495-1505 (electroporation); LI et al., (1993) Plant Cell Reports 12:250-255 and Christou and Ford (1995) Annals of Botany 75:745-750 (maize via Agrobacterium tumefaciens); all of which are herein incorporated by reference.

The cells, which have been transformed, may be grown into plants in accordance with conventional ways. See, for example, McCormick et al. (1986) Plant Cell Reports, 5:81-84. These plants may then be grown, and either pollinated with the same transformed strain or different strains, and the resulting hybrid having the desired phenotypic characteristic identified. Two or more generations may be grown to ensure that the subject phenotypic characteristics is stably maintained and inherited and then seeds harvested to ensure the desired phenotype or other property has been achieved. One of skill will recognize that after the recombinant expression cassette is stably incorporated in transgenic plants and confirmed to be operable, it can be introduced into other plants by sexual crossing. Any of number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plans that would produce the selected phenotype.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating(selling) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Backcrossing to a parental plant and out-crossing with a non-transgenic plant are also contemplated.

B. Transfection of Prokaryotes, Lower Eukaryotes, and Animal Cells

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc (1997).

The WRKY Transcriptional Regulatory Region

The transcriptional region for WRKY genes may be generally isolated from the 5′ untranslated region flanking their respective transcription initiation sites. Methods for isolation of transcriptional regulatory regions are well known in the art. By “isolated” is intended that the transcriptional regulatory region sequences have been determined and can be extracted by molecular techniques or synthesized by chemical means. In either instance, the transcriptional regulatory region is removed from at least one of its flanking sequences in its native state. The sequence for the transcriptional regulatory region of sunflower WRKY1-2 can be found in SEQ ID NO: 35.

It is recognized that regions in addition to the transcriptional regulatory region may be used to initiate transcription. Such regions include the UTR and even portions of the coding sequence particularly 5′ portions of the coding region. Generally, from about 3 nucleotides (1 codon) up to about 150 nucleotides (50 codons) of the 5′ coding region can be used. See, for example, McElroy et al. (1991) Mol Gen. Genet. 231: 150-160 and herein incorporated by reference, where expression vectors were constructed based on the rice actin 1 5′ region.

Comparable transcriptional regulatory regions from other plants may be obtained by utilization of the coding or promoter sequences of the invention. Using the WRKY coding sequences, other WRKY transcriptional regulatory regions can be isolated by obtaining regions 5′ to the regions of homology.

Methods are readily available in the art for the hybridization of nucleic acid sequences. Promoter sequences from other plants may be isolated according to well-known techniques based on their sequence homology to the promoter sequences set forth herein. In these techniques, all or part of the known transcriptional regulatory region sequence is used as a probe, which selectively hybridizes to other sequences present in a population of cloned genomic DNA, fragments (i.e.genomic libraries) from a chosen organism.

For example, the entire transcriptional regulatory region or portions thereof may be used as probes capable of specifically hybridizing to corresponding promoter sequences. To achieve specific hybridization under a variety of conditions, such probes include sequences that are unique and are preferably at least about 10 nucleotides in length, and most preferably at least about 20 nucleotides in length. Such probes may be used to amplify corresponding promoter sequences from a chosen organism by the well-known process of polymerase chain reaction (PCR). This technique may be used to isolate additional promoter sequences from a desired organism or as a diagnostic assay to determine the presence of the promoter sequence in an organism. Such techniques include hybridization screening of plated DNA libraries (either plaques or colonies; see e.g. Innis et al. (1990) PCR Protocols. A Guide to Methods and Applications, eds., Academic Press).

The isolated transcriptional regulatory region of the present invention can be modified to provide for a range of expression levels of the heterologous nucleotide sequence. Thus, less than the entire region may be utilized and the ability to drive pathogen or chemical-inducible expression retained. However, it is recognized that expression levels of mRNA may be altered and usually decreased with deletions of portions of the region. Generally, at least about 20 nucleotides of an isolated region will be used to drive expression of a nucleotide sequence.

It is recognized that to increase transcription levels enhancers may be utilized in combination with the promoter regions of the invention. Enhancers are nucleotide sequences that act to increase the expression of a promoter region. Enhancers are known in the art. For example, the enhancer from the cauliflower mosaic virus (CaMV) 35S promoter has been isolated.

Modifications of the isolated transcriptional regulatory region of the present invention can provide for a range of expression of the heterologous nucleotide sequence. Thus, they may be modified to be weak promoters or strong promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1000 transcripts.

The nucleotide sequences for the transcriptional regulatory region of the present invention may be the naturally occurring sequences or sequences having substantial homology. By “substantial homology” is intended a sequence exhibiting substantial functional and structural equivalence with the naturally occurring sequence. Any structural differences between substantially homologous sequences do not affect the ability of the sequence to function as a promoter as disclosed in the present invention. Thus, sequences having substantial sequence homology with the sequence of the transcriptional regulatory region of the present invention will direct expression during pathogen infection or chemical induction of an operably linked heterologous nucleotide sequence. Two transcriptional regulatory nucleotide sequences are considered substantially homologous when they have at least about 70%, preferably at least about 80%, more preferably at least about 90%, still more preferably at least about 95% sequence homology. Substantially homologous sequences of the present invention include variants of the disclosed sequences such as those that result from site-directed mutagenesis, as well as synthetically derived sequences.

Substantially homologous sequences of the present invention also refer to those fragments of a particular promoter nucleotide sequence disclosed herein that operate to promote the pathogen or chemical-inducible expression of an operably linked heterologous nucleotide sequence. These fragments will comprise at least about 20 contiguous nucleotides, or preferably 25, 30, 35, 40, 45, 50, 55, 60, 65, 70, 75, 80, 85, 90, 95, 100, 200, 300, 400, 500, 600, 700, 800, 900 or 1000 nucleotides of the transcriptional regulatory region of the present invention. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al. (1987) Methods Enzymol 155: 335-350, and Erlich, ed. (1989) PCR Technology (Stockton Press, New York). Again, variants of these transcriptional regulatory region fragments, such as those resulting from site-directed mutagenesis, are encompassed by the compositions of the present invention.

Nucleotide sequences comprising at least about 20 contiguous nucleotides of the sequence set forth in SEQ ID NO: 35 are encompassed. These sequences may be isolated by hybridization, PCR, and the like. Such sequences encompass fragments capable of driving developmentally regulated expression, fragments useful as probes to identify similar sequences, as well as elements responsible for temporal or tissue specificity. Biologically active variants of the promoter sequences are also encompassed by the method of the present invention. Such variants should retain promoter activity, particularly the ability to drive expression during flowering. Biologically active variants include, for example, the native promoter sequences of the invention having one or more nucleotide substitutions, deletions or insertions. Promoter activity may be measured by Northern blot analysis, reporter activity measurements when using transcriptional fusions, and the like. See, for example, Sambrook et al. (1989) Molecular Cloning: A Laboratory Manual (2^{nd} ed. Cold Spring Harbor Laboratory, Cold Spring Harbor, N.Y.), herein incorporated by reference.

The coding sequence expressed by the transcriptional regulatory region of the invention may be used for expressing proteins during pathogen infection or upon chemical induction with compounds such as oxalic acid or salicylic acid. The affect of various expressed proteins of interest include but are not limited to resistance to insects, resistance to disease, resistance to stress, agronomic traits and the like.

These results can be achieved by providing expression of heterologous or increased expression of endogenous products in the plant. Alternatively, the results can be achieved by providing for a reduction of expression of one or more endogenous products, particularly enzymes and cofactors in the plant. These changes result in a change in phenotype of the transformed plant. For example, the transcriptional regulatory regions of the invention can be used to express degradative enzymes that are degrade toxins used by pathogens for invasion of a plant. Alternatively, the transcriptional regulatory sequences of the invention can be used to produce antisense mRNA complementary to the coding sequence of an essential protein, inhibit production of a native protein that is required or promotes pathogen invasion.

General categories of genes of interest for the purposes of the present invention include for example, those genes involved in information, such as Zinc fingers, those involved in communication, such as kinases, and those involved in housekeeping, such as heat shock proteins. It is recognized that the genes of interest depend on the exact specificity of the WRKY transcriptional regulatory region.

More specific categories of transgenes, for example, include genes involved in flowering; genes involved in resistance to disease, pesticides and insect pests. It is recognized that any gene of interest can be operably linked to the promoter of the inventions and expressed during pathogen infection or upon chemical induction.

Genes involved in resistance to insects may encode resistance to insect pests such as second generation corn borer (Ostinia nubilalis) and adult rootworm beetle (Diabrotica virgifera). Such genes include, for example, Bacillus thuringiensis endotoxin genes (U.S. Pat. Nos. 5,366,892; 5,747,450; 5,737,514; 5,723,756; 5,593,881; Geiser et al., Gene 48:109 (1986); lectins (Van Damme et al., Cell 78:1089 (1994); and the like.

Gene encoding resistance to disease traits may include detoxification genes, against fumonisin (U.S. Pat. Nos. 5,792,931 and 5,716,820); oxalate decarboxylase (PCT patent publication No. 98/42827); oxalate oxidase (PCT publication No. WO 92/14824 and PCT publication WO 92/15685); glucose oxidase (U.S. Pat. No. 5,516,671); avirulence (avr) and disease resistance (R) genes (Jones et al., Science 266:789 (1994); Martin et al., Science 262:1432 (1993); Mindrinos et al., Cell 78:1089 (1994)); and the like.

Exogenous products include plant enzymes and products as well as those from other sources including prokaryotes and other eukaryotes. Such products include enzymes, cofactors, hormones, and the like.

The heterologous nucleotide sequence operably linked to one of the promoters disclosed herein may be an antisense sequence for a targeted gene. By “antisense DNA nucleotide sequence” is intended a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is invited to achieve a desired phenotypic response. Thus, the promoter sequences disclosed herein may be operably linked to antisense DNA sequence to reduce or inhibit expression of a native protein in the plant.

Modulating polypeptide Levels and/or Composition

The present invention further provides a method for modulating (i.e., increasing or decreasing) the concentration or composition of the polypeptides of the present invention in a plant or part thereof. Increasing or decreasing the concentration and/or the composition (i.e., the ratio of the polypeptides of the present invention) in a plant can effect modulation. The method comprised introducing into a plant cell with a recombinant expression cassette comprising a polynucleotide of the present invention as described above to obtain a transformed plant cell, culturing the transformed plant cell under plant cell growing conditions, and inducing or repressing expression of a polynucleotide of the present invention in the plant for a time sufficient to modulate concentration and/or composition in the plant or plant part.

In some embodiments, the content and/or composition of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. No. 5,565,350; Zarling et al., PCT/US93/03868. And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art and discussed briefly, supra.

In general, concentration or composition is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra Induction of expression of a polynucleotide of the present invention can also be controlled by exogenous administration of an effective amount of inducing compound. Inducible promoters and inducing compounds, which activate expression from these promoters, are well known in the art. In preferred embodiments, the polypeptides of the present invention are modulated in monocots, particularly maize.

Molecular Markers

The present invention provides a method of genotyping a plant comprising a polynucleotide of the present invention. Optionally, the plant is a monocot, such as maize or sorghum. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson 1996 (Chapter 2) in: Genome Mapping in plants (ed. Andrew H. Paterson) by Academic Press/R. G. Lands Company, Austin, Tex., pp. 7-21.

The particular method of genotyping in the present invention may employ any number of molecular marker analytic techniques such as, but not limited to, restriction fragment length polymorphism's (RFLPs). RFLPs are the product of allelic differences between DNA restriction fragments resulting from nucleotide sequence variability. As is well known to those of skill in the art, RFLPs are typically detected by extraction of genomic DNA and digestion with a restriction enzyme. Generally, the resulting fragments are separated according to size and hybridized with a probe; single copy probes are preferred. Restriction fragments from homologous chromosomes are revealed. Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, preferably within 20 or 10 cM, more preferably within 5, 3, 2, or 1 cM of a gene of the present invention.

In the present invention, the nucleic acid probes employed for molecular marker mapping of plant nuclear genomes selectively hybridize, under selective hybridization conditions, to a gene encoding a polynucleotide of the present invention. In preferred embodiments, the probes are selected from polynucleotides of the present invention. Typically, these probes are cDNA probes or restriction enzyme treated (e.g., PST 1) genomic clones. The length of the probes is discussed in greater detail, supra, but is typically at least 15 bases in length, more preferably at least 20, 25, 30, 35, 40, or 50 bases in length. Generally, however, the probes are less than about 1 kilobase in length. Preferably, the probes are single copy probes that hybridize to a unique locus in haploid chromosome compliment. Some exemplary restriction enzymes employed in RFLP mapping are EcoRI, EcoRv, and SstI. As used herein the term “restriction enzyme” includes reference to a composition that recognizes and, alone or in conjunction with another composition, cleaves at a specific nucleotide sequence.

The method of detecting an RFLP comprises the steps of (a) digesting genomic DNA of a plant with a restriction enzyme; (b) hybridizing a nucleic acid probe, under selective hybridization conditions, to a sequence of a polynucleotide of the present of said genomic DNA; (c) detecting therefrom a RFLP. Other methods of differentiating polymorphic (allelic) variants of polynucleotides of the present invention can be had by utilizing molecular marker techniques well known to those of skill in the art including such techniques as: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC). Thus, the present invention further provides a method of genotyping comprising the steps of contacting, under stringent hybridization conditions, a sample suspected of comprising a polynucleotide of the present invention with a nucleic acid probe. Generally, the sample is a plant sample, preferably, a sample suspected of comprising a maize polynucleotide of the present invention (e.g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In preferred embodiments, the nucleic acid probe comprises a polynucleotide of the present invention.

UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, Nucleic Acids Res 15:8125 (1987)) and the 7-methylguanosine cap structure (Drummond et al., Nucleic Acids Res. 13:7375 (1985)). Negative elements include stable intramolecular 5′ UTR stem-loop structures (Muesing et al., Cell 48:691 (1987)) and AUG sequences or short open reading frames preceded by an appropriate AUG in the 5′ UTR (Kozak, supra, Rao et al., Mol. and Cell. Biol. 8:284 (1988)). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host such as to optimize the codon usage in a heterologous sequence for expression in maize. Codon usage in the coding regions of the polynucleotides of the present invention can be analyzed statistically using commercially available software packages such as “Codon Preference” available form the University of Wisconsin Genetics Computer Group (see Devereaux et al., Nucleic Acids Res. 12:387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

Sequence Shuffling

The present invention provides methods for sequence shuffling using polynucleotides of the present invention, and compositions resulting therefrom. Sequence shuffling is described in PCT publication No. WO 96/19256. See also, Zhang, J. -H., et al. Proc. Natl. Acad. Sci. USA 94:4504-4509 (1997). Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Kₘ and/or increased K_{cat} over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140%, or at least 150% of the wild-type value.

Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide, sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phylums, or kingdoms. For example, a polynucleotide having a consensus sequence from a gene family of Zea mays can be used to generate antibody or nucleic acid probes or primers to other Gramineae species such as wheat, rice, or sorghum. Alternatively, a polynucleotide having a consensus sequence generated from orthologous genes can be used to identify or isolate orthologs of other taxa. Typically, a polynucleotide having a consensus sequence will be at least 9, 10, 15, 20, 25, 30, or 40 amino acids in length, or 20, 30, 40, 50, 100, or 150 nucleotides in length. As those of skill in the art are aware, a conservative amino acid substitution can be used for amino acids, which differ amongst aligned sequence but are from the same conservative amino substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in chapter 7 of Current Protocols in Molecular Biology, F. M. Ausubel et al., Eds. Current Protocols, a joint venture between Greene Publishing Associates, Inc. and John Wiley & Sons, Inc. (Supplement 30). A polynucleotide sequence is considered similar to a reference sequence if the smallest sum probability in a comparison of the test nucleic acid to the reference nucleic acid is less then about 0.1, more preferably less than about 0.01, or 0.001, and most preferably less than about 0.0001, or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCER. Conveniently, default parameters of such software can be used to generate consensus or generic sequences.

Use of Subsequences of WRKY Polynucleotides

As previously discussed, WRKY polynucleotides have conserved domains. The binding specificity of the WRKY domains is a hallmark of a specific set of promoters that a particular WRKY interacts with. Therefore, a subsequence of a WRKY polynucleotide could be utilized in the following manner.

First, a subsequence of WRKY could be expressed in an expression system (please see the section entitled “Expression of Proteins in Host Cells”), such as an E. coli expression system. The ability of the expressed protein could then be tested for its ability to bind target DNA in a gel shift experiment or other interaction assay. Either specific candidate promoter DNA or total genomic DNA could be used in the experiment.

Alternatively, a subsequence of a WRKY polynucleotide could be fused in frame to an N-terminal DNA activation domain, such as, but not limited to, a myb or myc homolog or the activation domain of another WRKY. The fusion polynucleotide would then be expressed in an expression system, such as, but not limited to, a transient or stable plant expression system. Specific promoters could then be identified or global transcript profiling could be used to identify genes and their associated promoters that respond to the WRKY domain/activation domain fusion.

Although the present invention has been described in some detail by way of illustration and example for purposes of clarity of understanding, it will be obvious that certain changes and modifications may be practices within the scope of the appended claims.

### EXAMPLE 1

### Isolation of Maize ZmWRKY3-1 cDNA

Using the techniques described above a partial sequence of a homolog of parsley WRKY3 was found in a maize cDNA library. A cDNA library was made from mRNA isolated from maize cells. The maize cells were treated with water or 1×10⁶ spores/ml of Fusarium moniliforme. Cells were harvested 2 and 6 hours after treatment. Total RNA was isolated using Tri-Reagent™ and mRNA was isolated using PolyAtract™ (Promega). Zap-cDNA synthesis kit (Stratagene) was used to prepare cDNA, which was cloned into HybriZap® (Stratagene). The primary library was amplified and phagemid was excised from the secondary library. The phagemid prep was amplified in XLOLR cells and purified (Qiagen). All library manipulations were performed according to the HybriZap® manual.

The full-length sequence was cloned from the lambda cDNA library screen using typical plaque hybridization techniques found in Sambrook et al., Molecular Cloning—A Laboratory Manual, 2nd ed., Vol. 1-3 (1989). The nucleic acid sequence and amino acid sequence of ZmWRKY3-1 can be found in SEQ ID NOS: 1 and 2, respectively.

Gene identities can be determined by conducting BLAST (Basic Local Alignment Search Tool; Altschul, S. F., et al., (1993) J. Mol. Biol. 215:403-410; see also www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences are analyzed for similarity to all publicly available DNA sequences contained in the “nr” database using the BLASTN algorithm. The DNA sequences are translated in all reading frames and compared for similarity to all publicly available protein sequences contained in the “nr” database using the BLASTX algorithm (Gish, W. and States, D. J. Nature Genetics 3:266-272 (1993)) provided by the NCBI. In some cases, the sequencing data from two or more clones containing overlapping segments of DNA are used to construct contiguous DNA sequences.

Additional maize WRKY sequences were identified from a cDNA library generated and sequenced as described below. Total RNA was isolated from corn tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples were pulverized in liquid nitrogen before the addition of the TRIzol Reagent, and then were further homogenized with a mortar and pestle. Addition of chloroform followed by centrifugation was conducted for separation of an aqueous phase and an organic phase. The total RNA was recovered by precipitation with isopropyl alcohol from the aqueous phase.

The selection of poly(A)+ RNA from total RNA was performed using PolyATact system (Promega Corporation, Madison Wis.). In brief, biotinylated oligo(dT) primers were used to hybridize to the 3′ poly(A) tails on mRNA. The hybrids were captured using streptavidin coupled to paramagnetic particles and a magnetic separation stand. The mRNA was washed at high stringent condition and eluted by RNase-free deionized water.

cDNA synthesis was performed and unidirectional cDNA libraries were constructed using the SuperScript Plasmid System (Life Technology Inc. Gaithersburg, Md.). The first strand of cDNA was synthesized by priming an oligo(dT) primer containing a Not I site. The reaction was catalyzed by SuperScript reverse Transcriptase II at 45° C. The second strand of cDNA was labeled with alpha-³²P-dCTP and a portion of the reaction was analyzed by agarose gel electrophoresis to determine cDNA sizes. cDNA molecules smaller than 500 base pairs and unligated adaptors were removed by Sephacryl-S400 chromatography. The selected cDNA molecules were ligated into a pSPORT1 vector between the NotI and SalI sites.

Individual colonies were picked and DNA was prepared either by PCR with Ml 3 forward primers and M13 reverse primers, or by plasmid isolation. All the cDNA clones were sequenced using M13 reverse primers.

cDNA libraries subjected to the subtraction procedure were plated out on 22×22 cm² agar plate at density of about 3,000 colonies per plate. The plates were incubated in a 37° C. incubator for 12-24 hours. Colonies were picked into 384-well plates by a robot colony picker, Q-bot (GENETIX Limited). These plates were incubated overnight at 37° C.

Once sufficient colonies were picked, they were pinned onto 22×22 cm² nylon membranes using Q-bot. Each membrane contained 9,216 colonies or 36,864 colonies. These membranes were placed onto agar plate with appropriate antibiotic. The plates were incubated at 37° C. for overnight.

After colonies were recovered on the second day, these filters were placed on filter paper prewetted with denaturing solution for four minutes, then were incubated on top of a boiling water bath for additional four minutes. The filters were then placed on filter paper prewetted with neutralizing solution for four minutes. After excess solution was removed by placing the filters on dry filter papers for one minute, the colony site of the filters were placed into Proteinase K solution, incubated at 37° C. for 40-50 minutes. The filters were placed on dry filter papers to dry overnight. DNA was then cross-linked to nylon membrane by UV light treatment.

Colony hybridization was conducted as described by Sambrook, J., Fritsch, E. F. and Maniatis, T., (in Molecular Cloning: A laboratory Manual, 2^{nd} Edition). The following probes were used in colony hybridization:

1. First strand cDNA from the same tissue as the library was made from to remove the most redundant clones.

2. 48-192 most redundant cDNA clones from the same library based on previous sequencing data.

3. 192 most redundant cDNA clones in the entire corn sequence database.

4. A Sal-A20 oligo nucleotide TCG ACC CAC GCG TCC GAA AAA AAA AAA AAA AAA AAA, (SEQ ID NO: 36) removes clones containing a poly A tail but no cDNA.

5. cDNA clones derived from rRNA.

The image of the autoradiography was scanned into computer and the signal intensity and cold colony addresses of each colony was analyzed, re-arraying of cold-colonies from 384 well plates to 96 well plates was conducted using Q-bot. The cDNA sequence information generated from the cDNA library was then analyzed by BLAST to find additional maize WRKY polynucleotides.

The following maize WRKY polynucleotides were found as described above. ZmWRKY1-1 polynucleotide is shown in SEQ ID NO: 37. The protein translation of ZmWRKY1-1 is shown in SEQ ID NO: 38. The ZmWRKY1-2 polynucleotide is shown in SEQ ID NO: 39. The ZmWRKY2-2 polynucleotide is shown in SEQ ID NO: 40. The ZmWRKY3-3 polynucleotide is shown in SEQ ID NO: 41. The ZmWRKY3-4 polynucleotide is shown in SEQ ID NO: 42. The ZmWRKY3-5 polynucleotide is shown in SEQ ID NO: 43.

Northern Blot Assay

The mRNA steady-state level of maize WRKY1 and WRKY3 were studied after treatment with Fusarium moniliforme spores. Mid-log maize GS3 suspension cell cultures (75 ml) were treated with 1 ml of Fusarium spores to give a concentration of 1,000,000 spores/ml. Control cultures were treated with 1 ml of water. The cultures were harvested at 0, 1, and 3 hours post-treatment. RNA was extracted and Northern Blot analysis was performed according to Church, et al., Proc. Natl. Acad. Sci. USA 81:1991-1995 (1984). The blots were probed with DNA that was either ZmWRKY1-(SEQ ID NO: 37) or ZmWRKY3-1 (SEQ ID NO: 1). At 1 and 3 hours post-treatment there was a significant induction of both ZmWRKY1-1 and ZmWRKY3-1, substantiating the role of ZmWRKY1-1 and ZmWRKY3-1 in a plants response to pathogen infection.

Transgenic Evaluation of ZmWRKY3-1

The promoter region of ZmPR-1 gene (PCT Publication WO 99/43819) was fused with the coding sequence of a β-glucuronidase (GUS) reporter gene resulting in a molecular marker construct (ZmPR-1::GUS). The coding sequences of ZmNPR1 (PCT Publication number WO 00/65037) and ZmWRKY3-1 driven by the ubiquitin promoter were employed as regulator constructs (Ubi::ZmNPR1 and Ubi::ZmWRKY3). Act::luciferase (rice actin promoter (U.S. Pat. No. 5,641,876) operably linked to the luciferase gene from the Promega Dual-luciferase reporter assay system) was used as an internal standard for normalization of the variation inherent in bombardment. A DNA carrier construct was also included to maintain uniform DNA concentrations.

Maize immature embryos (IE) were co-bombarded with the marker construct and either the DNA carrier construct or the regulator construct. The internal standard was also included in all bombardments. Mixture of DNA from 20 μl of ZmPR-1::GUS at 0.05 μg/μl, 5 μl of the regulator or carrier DNA (1.0 μg/μl), and 10 μl of Act::luciferase at 0.1 μg/μl were co-precipitated with 70 μl of 2.5 M CaC1₂ and 20 μl of 0.1 M spermidine onto 50 μl of tungsten particles (1.0 μm at a particle density of 15 mg/ml). For each bombardment, 45 IEs were placed on a high osmotic medium (12 g/L sucrose) plate for 4 hours before the bombardment. After the bombardment the IEs were placed in culture on the same osmotic medium for 24 hours and then divided into three groups. One group was cultured on a piece of filter paper wetted with the same osmotic medium without any addition of signal molecules as a control and the other two were cultured under the same condition but the medium contained either 1 mM SA or 0.1 mM JA. All IEs were cultured for another 24 hours.

Three IEs from each group were histochemically stained in X-Gluc staining solution for overnight at 37° C. The rest of the IEs were subjected to GUS fluorometric and luciferase assays. Fluorometric measurements of GUS activity were performed by using 50 μl protein extract prepared from the 12 IEs of each treatment and quantified in Fluoroskan Ascent FL (Labsystem) for two time points, 10 and 30 min. Luciferase activity was quantified in a Monolight 2010 (Analytical Luminescence Lab) by mixing 20 μl of protein extract with 100 μl of reaction buffer (Dual-Luciferase Reporter Assay System, Promega) and taking the measurements after 10 seconds. To normalize promoter/marker activity, the GUS value detected in each sample was divided by the luciferase value obtained in the same bombarded sample treated without signal molecules.

It has been established in Arabidopsis that SA and NPR1 are two key regulators that activate the SA-dependent SAR response. Both histochemical and fluorometric GUS assay results showed that ZmPR-1::GUS expression was induced by more than 3-fold by SA treatment alone, as well as in cells over-expressing ZmNPR1 alone.

In contrast, cells expressing WRKY3-1 showed complete suppression of GUS activity under both JA treatment and no treatment. An antagonistic relationship between the SA- and JA-dependent plant defense signaling transduction pathways has been shown in several reports. WRKY factors have been proposed as repressors of PR-1 expression. The results indicate that JA and ZmWRKY3-1 suppress ZmPR-1::GUS expression in maize. Thus, ZmWRKY3-1 functions in suppression of ZmPR-1 in a transient system. This suppression of ZmPR-1 is consistent with what is expected for at least certain WRKY genes and is a further indicator of the role ZmWRKY3-1 plays in a plant's defense to disease.

Therefore, to modulate the level of disease resistance in a plant using a WRKY polynucleotide, it may be necessary to inhibit or lower the expression of the native WRKY gene or in the alternative increase expression by overexpression of the transgene, depending the disease resistance pathway to be modified. Methods of decreasing expression of a gene in a plant are well known in the art. For example, reduction in the expression of a WRKY gene can be accomplished by a number of methods, including but not limited to, antisense, catalytic RNA molecules (ribozymes), cross-linking agents, alkylating agents, radical generating species, or sense suppression. A discussion of these methods can be found in the section entitled “Recombinant Expression Cassettes.” If suppression of WRKY is only desired during pathogen infection, then a pathogen inducible promoter operably linked to the WRKY polynucleotide in the sense orientation for sense suppression or antisense orientation for antisense suppression may be used. Alternatively a constitutive promoter operably linked to a WRKY polynucleotide in the sense or antisense orientation may be used. The recombinant expression cassette can then be transformed into plant cells and a whole plant can be regenerated.

Alternatively, the native WRKY gene can be modified by chimeric oligonucleotides. U.S. Pat. No. 5,565,350 describes chimeric oligonucleotides that are useful for targeted gene correction and methods for their use in cultured mammalian cells. The use of chimeric oligonucleotides in plants is described in PCT Publication No. WO 99/25853, published May 27, 1999. Both disclosures are herein incorporated by reference.

In addition, the expression of WRKY gene may be reduced by the use of hairpin dsRNA techniques. These techniques are illustrated in PCT published applicant No. WO 99/53050, published Oct. 21, 1999 and WO 98/53083 published Nov. 26, 1998, both of which are herein incorporated by reference.

### EXAMPLE 2

### Isolation of Sunflower WRKY Polynucleotides (SWRKY1)

Fungal Infection and Chemical Treatments:

Sunflower plants (SMF3) were planted in 4-inch pot and grown in greenhouse for first four weeks. After transfer to growth chamber, plants were maintained under a 12-hour photoperiod at 22° C. with an 80% relative humidity. Six-week old plants were inoculated with Sclerotinia-infected carrot plugs or sprayed with four different chemicals at the given concentration. For each plant, three petioles were inoculated and wrapped with lx2 inch parafilm. Plant tissue samples were harvested at different time points and immediately frozen in liquid nitrogen and then stored at −80° C.

Construction of the Sclerotinia-infected and Resistance-enhanced Sunflower cDNA Libraries:

Six-week old SMF3 sunflower plants were infected with Sclerotinia sclerotrium by petiole inoculation with Sclerotinia-infested carrot plugs. Six days after infection, leaf and stem tissues were collected from infected plants for total RNA isolation. Total RNA was also isolated from transgenic sunflower plants expressing a wheat oxalate oxidase gene at the 6-week stage (U.S. Pat. No. 6,166,291; and hereby incorporated by reference). Previous studies have showed that elevated levels of H₂O₂, SA and PR1 protein were detected in oxalate oxidase expressing transgenic plants at the 6-week stage and that the plants showed more resistant to Sclerotinia infection (U.S. Pat. No. 6,166,291). The mRNAs were isolated by a mRNA purification kit (BRL) according to manufacture's instruction. The cDNA libraries were constructed with the ZAP-cDNA synthesis kit into pBluescript phagemid (Stratagene). A cDNA library mixture for PCR cloning was made of oxalate oxidase transgenic stem and Sclerotinia-infected leaf libraries (1:2 mix).

PCR amplification of Sunflower WRKY Genes:

To isolate sunflower WRKY genes, a conserved motif (WRKYGQK) of zinc-finger type transcriptional factor was used to design four degenerate primers:

W-s1: 5′-TGGMGNAARTAYGGNCAGAA-3′ (SEQ ID NO: 3)

W-s2: 5′-TGGMGNAARTAYGGNCAAAA-3′ (SEQ ID NO: 4)

W-as1: 5′-TTYTGNCCRTAYTTNCGCCA-3′ (SEQ ID NO: 5)

W-as2: 5′-TTYTGNCCRTAYTTNCTCCA-3′ (SEQ ID NO: 6)

Primers for Library Vector (pBS)

PBS-upper: GCGATTAAGTTGGGTAACGCCAGGGT (SEQ ID NO: 7)

PBS-lower: TCCGGCTCGTATGTTGTGTGGAATTG (SEQ ID NO: 8)

The cDNA library was used as the DNA template for PCR amplification. To facilitate the cloning process, a pair of 28 base pair vector primers of flanking cDNA (3′ and 5′) of pBS vector were designed. The primers were directionally amplified with either the 5′ or 3′ end of the cDNA of the vector primers (pBS-upper or pBS-lower) paired with a degenerate primer. The full-length cDNA was amplified using a new gene specific primer containing the region upstream of the ATG start sequence and the vector primer at the 3′ end.

PCR reactions were performed in a total volume of 25 ul in 10 mM Tris—HCl, pH 8.3; 1.5 mM MgCL₂; 50 mM KCl; 0.1 mM dNTPs; 0.25 μM of each primer with 0.5 units of advantage cDNA polymerase mix (Clontech) or Pwo DNA polymerase (Boehringer Mannheim). Genomic DNA and/or cDNA library mixtures were used as templates for PCR amplification.

Analysis of Amplified PCR Products:

Amplified PCR fragments with the expected sizes were individually sliced out of the gel for a second round of PCR re-amplification with the same condition as initial PCR. Each second round of PCR product showing a single band with the expected size was cloned into a TA vector (Clontech) according to the supplier's instructions. Positive clones were sequenced using an Applied Biosystems 373A automated sequencer. DNA sequence analysis was carried out with Sequencer (3.0). Multiple-sequence alignments of the DNA sequence were carried out using CLUSTAL W (Thompson, et al., Nuc. Acids Res. 22:4673-80 (1994)).

Results

Four sunflower WRKY homologs have been cloned and sequenced. The SWRKY1-1 polynucleotide and polypeptide sequence is shown in SEQ ID NOS: 9 and 10. SWRKY1-2 polynucleotide and polypeptide sequence is shown in SEQ ID NOS: 11 and 12. SWRKY1-3 polynucleotide and polypeptide sequence is shown in SEQ ID NOS: 13 and 14. SWRKY1-4 polynucleotide and polypeptide sequence is shown in SEQ ID NOS: 15 and 16. BLAST search results indicates that all four cDNAs were homologous to parsley WRKY1 gene. Amino acid sequence alignment and genetic distance analysis reveals that three of the sunflower WRKY genes (SWRKY1-3, 1-2 and 1-4) are very closely related. Sunflower WRKY1-1 is less similar to the other sunflower WRKY genes but is closer in homology to the parsley WRKY1 gene.

Northern Blot Assay

The mRNA steady-state level of sunflower WRKY1 was studied under different chemical treatments. Six-week-old sunflower plants were sprayed with oxalic acid (OA) (5 mM), hydrogen peroxide (5 mM), salicylic acid (SA) (5 mM) and jasmonic acid (JA) (45 uM in 0.1% ethanol). Leaf samples were collected at 0, 6, 12, and 24 hours after application and immediately frozen in liquid nitrogen. Twenty microgram of total RNA were loaded in each sample lane. Control tissue was SMF3 leaf tissue with no treatment. Northern Blot analysis was performed according to Church, et al., Proc. Natl. Acad Sci. USA 81:1991-1995 (1984). The blots were probed with DNA from the sunflower WRKY1-1 polynucleotide. The salicylic acid and oxalic acid treatments showed significant induction of WRKY1-1 within 6 hours. The hydrogen peroxide and jasmonic acid treatments did not induce WRKY1-1 RNA within 6 hours.

The mRNA steady-state level of sunflower WRKY1 gene was also studied under Sclerotinia-infection and oxalate oxidase expression. Six-week-old transgenic sunflower leaf and stem samples were collected along with control SMF3 samples. Sclerotinia-infected samples were harvested on 6 days after inoculation. Twenty microgram of total RNA were loaded in each sample lane. Northern Blot analysis was performed according to Church, et al., Proc. Natl. Acad Sci. USA 81:1991-1995 (1984). The blots were probed with sunflower WRKY1-1 polynucleotide. Sunflower WRKY1-1 was induced by Sclerotinia infection and oxalate oxidase expression in sunflower.

Isolation of Disease Inducible Transcriptional Regulatory Regions:

The 5′-flanking regulatory region of WRKY1-2 (SEQ ID NO: 35) was isolated from sunflower genomic DNA using Universal GenomeWalker Kit (Clontech) according to the manufacturer instruction. Sunflower inbred line SMF3 was grown in the greenhouse and growth chamber. Mature leaf tissue from the sunflower line SMF 3 was used for genomic DNA isolation. (Rogers, et al., (1994) Extraction of total cellular DNA from plants, algae and fungi. In Plant Molecular Biology Manual (eds. Gelvin, S. B. and Schilperoort. second edition). Restriction digested genomic DNAs were ligated with an adaptor to construct pools of genomic DNA fragments (GenomeWalker libraries) for walking by PCR. (Siebert et al., Nuc. Acids Res. 23:1087-1088 (1995)).

PCR reactions were performed in a total volume of 25 ul in 10 mM Tris—HCL, pH 8.3; 1.5 mM MgCL2; 50 mM KCL; 0.1 mM dNTPs; 0.25 uM of each primer with 0.5 units DNA polymerase (Clontech). GenomicWalker libraries were used as template for PCR amplification.

Amplified PCR fragments with the expected sizes were individually sliced out of the gel for a second round PCR re-amplification with the same condition as the initial PCR. Each second round PCR product showing a single band with the expected size was cloned into TA vector (Invitrogen) according to the supplier's instructions. Identified positive clones were selected for DNA sequencing using an Applied Biosystems 373A (ABI) automated sequencer. DNA sequence analysis was carried out with Sequencer (3.0).

### EXAMPLE 3

### Isolation of Rice WRKY, Soybean WRKY, Wheat WRKY and Other Maize WRKY Polynucleotides

Composition of cDNA Libraries: Isolation and Sequence of cDNA Clones

For cDNA libraries various tissues were prepared. The characteristics of the libraries are described below.

TABLE 1
cDNA Libraries

| Library | Tissue | Clone |
|---|---|---|
| rls24 | Rice (Oryza sativa L.) leaf (15 DAG) 24 hours after | rls24.pk0005.d1 |
| | infection of strain 4360-R-67 | |
| rdr1f | Rice (Oryza sativa L.), developing root of 10 day old | rdr1f.pk004.m4 |
| | plants, full length enriched library | |
| srr3c | Soybean (Glycine max L., Bell) roots | srr3c.pk001.a20 |
| sfl1 | Soybean, (Glycine max L.) immature flower | sfl1.pk0008.a2 |
| sdp4c | Soybean (Glycine max L.) developing pods, 10-12 mm. | spd4c.pk007.b19 |
| wlk4 | Wheat, (Triticum aestivum L.) seedlings 4 hours after | wlk4.pk0012.c10 |
| | treatment with the wheat fungicide KQ926 | |
| wlmk8 | Wheat (Triticum aestivum L.), seedlings 8 hours after | wlmk8.pk0019.b11 |
| | inoculation with Erysiphe graminis and treatment with | |
| | the wheat fungicide KQ926 | |
| cr1n | Maize (Zea mays), root tissue from 7 day old etiolated | crln.pk.0183.d7 |
| | seedlings | |
| cpk1c | Maize (Zea mays), pooled BMS, treated with chemicals | cpk1c.pk001.f20 |
| | related to membrane traffic | |

cDNA libraries were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (see Adams, M. D. et al., (1991) Science 252:1651). The resulting sequences were analyzed using a Perkin Elmer Model 377 fluorescent sequencer.

Characterization of cDNA Clones Encoding Rice WRKY1 and WRKY3

The BLASTX search using the sequences from clone r1s24.pk0005.d1 revealed similarity of the proteins encoded by the cDNAs to WRKY1 from Petroselinum crispum (NCBI Accession No. 1431872) with a pLog score of 26.22. The sequence of a portion of the cDNA insert from clone r1s24.pk0005.d1 is shown in SEQ ID NO: 17; the deduced amino acid sequence of this cDNA is shown in SEQ ID NO: 18. BLAST scores and probabilities indicate that the instant nucleic acid fragments encode portions of WRKY1. These sequences represent the first rice sequence encoding WRKY1.

The BLASTX search using the sequences from clone rdr1f.pk004.m4 revealed similarity of the proteins encoded by the cDNAs to WRKY3 from Avena sativa (NCBI Accession No. 4894963) with a pLog score of 28.00. The sequence of a portion of the cDNA insert from clone rdr1f.pk004.m4 is shown in SEQ ID NO: 19; the deduced amino acid sequence of this cDNA is shown in SEQ ID NO: 20. BLAST scores and probabilities indicate that the instant nucleic acid fragments encode portions of WRKY3. These sequences represent the first rice sequence encoding WRKY3.

Characterization of cDNA Clones Encoding Soybean WRKY1, WRKY2-1, and WRKY3

The BLASTX search using the sequences from clone srr3c.pk001.a20 revealed similarity of the proteins encoded by the cDNAs to WRKY1 from Nicotiana tabacum (NCBI Accession No. 5360683) with a pLog score of 28.40. The sequence of a portion of the cDNA insert from clone srr3c.pk001.a20 is shown in SEQ ID NO: 21; the deduced amino acid sequence of this cDNA is shown in SEQ ID NO: 22. BLAST scores and probabilities indicate that the instant nucleic acid fragments encode portions of WRKY1. These sequences represent the first soybean sequence encoding WRKY1.

The BLASTX search using the sequences from clone sfll.pk0008.a2 revealed similarity of the proteins encoded by the cDNAs to WRKY2 from Petroselinum crispum (NCBI Accession No. 1432058) with a pLog score of 70.70. The sequence of a portion of the cDNA insert from clone sfll.pk0008.a2 is shown in SEQ ID NO: 23; the deduced amino acid sequence of this cDNA is shown in SEQ ID NO: 24. BLAST scores and probabilities indicate that the instant nucleic acid fragments encode portions of WRKY2. These sequences represent the first soybean sequence encoding WRKY2.

The BLASTX search using the sequences from clone sdp4c.pk007.b19 revealed similarity of the proteins encoded by the cDNAs to WRKY3 from Nicotiana tabacum (NCBI Accession No. 4760596) with a pLog score of 28.10. The sequence of a portion of the cDNA insert from clone sdp4c.pk007.b19 is shown in SEQ ID NO: 25; the deduced amino acid sequence of this cDNA is shown in SEQ ID NO: 26. BLAST scores and probabilities indicate that the instant nucleic acid fragments encode portions of WRKY3. These sequences represent the first soybean sequence encoding WRKY3.

Characterization of cDNA Clones Encoding Wheat WRKY2 and WRKY3

The BLASTX search using the sequences from clone wlk4.pk0012.c10 revealed similarity of the proteins encoded by the cDNAs to WRKY2 from Nicotiana tabacum (NCBI Accession No. 4760692) with a pLog score of 87.70. The sequence of a portion of the cDNA insert from clone wlk4.pk0012.c10 is shown in SEQ ID NO: 27; the deduced amino acid sequence of this cDNA is shown in SEQ ID NO: 28. BLAST scores and probabilities indicate that the instant nucleic acid fragments encode portions of WRKY2. These sequences represent the first wheat sequence encoding WRKY2.

The BLASTX search using the sequences from clone wlmk8.pk0019.b11 revealed similarity of the proteins encoded by the cDNAs to WRKY3 from Avena sativa (NCBI Accession No. 4894963) with a pLog score of 148.00. The sequence of a portion of the cDNA insert from clone wlmk8.pkOOl9.bl 1 is shown in SEQ ID NO: 29; the deduced amino acid sequence of this cDNA is shown in SEQ ID NO: 30. BLAST scores and probabilities indicate that the instant nucleic acid fragments encode portions of WRKY3. These sequences represent the first wheat sequence encoding WRKY3.

Characterization of cDNA Clones Encoding Maize WRKY2-1 and WRKY3-2

The BLASTX search using the sequences from clone cr1n.pk0183.d7 revealed similarity of the proteins encoded by the cDNAs to WRKY2-1 from Petroselinum crispum (NCBI Accession No. 1432058) with a pLog score of 47.22. The sequence of a portion of the cDNA insert from clone cr1n.pk0183.d7 is shown in SEQ ID NO: 31; the deduced amino acid sequence of this cDNA is shown in SEQ ID NO: 32. BLAST scores and probabilities indicate that the instant nucleic acid fragments encode portions of WRKY2-1. These sequences represent the first maize sequence encoding WRKY2-1.

The BLASTX search using the sequences from clone cpk1c.pk001.f20 revealed similarity of the proteins encoded by the cDNAs to WRKY3 from Nicotiana tabacum (NCBI Accession No. 4760596) with a pLog score of 15.70. The sequence of a portion of the cDNA insert from clone cpk1c.pk001.f20 is shown in SEQ ID NO: 33; the deduced amino acid sequence of this cDNA is shown in SEQ ID NO: 34. BLAST scores and probabilities indicate that the instant nucleic acid fragments encode portions of WRKY3-2. These sequences represent the first maize sequence encoding WRKY3-2.

### EXAMPLE 4

Transformation and Regeneration of Transgenic Maize Plants

Immature maize embryos from greenhouse donor plants are bombarded with a plasmid containing a WRKY sequences of the present invention operably linked to a ubiquitin promoter and the selectable marker gene PAT (Wohlleben et al. (1988) Gene 70:25-37), which confers resistance to the herbicide Bialophos. Alternatively, the selectable marker gene is provided on a separate plasmid. Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

This plasmid DNA containing the WRKY polynucleotide plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialophos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for and altered level of expression of the WRKY sequence of the invention. Alternatively, the WRKY activity can be assayed (i.e., enhance disease resistance).

Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialophos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialophos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### EXAMPLE 5

### Agrobacterium-mediated Transformation of Maize

For Agrobacterium-mediated transformation of maize with a WRKY polynucleotide operably linked to ubiquitin promoter, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the WRKY nucleotide sequences to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### EXAMPLE 6

### Soybean Embryo Transformation

Soybean embryos are bombarded with a plasmid containing a WRKY polynucleotide operably linked to a Scp1 promoter (U.S. Pat. No. 6,072,050) as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the WRKY sequence operably linked to the Scpl promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

The above examples are provided to illustrate the invention but not to limit its scope. Other variants of the invention will be readily apparent to one of ordinary skill in the art and are encompassed by the appended claims. All publications, patents, and patent applications cited herein are indicative of the level of those skilled in the art to which this invention pertains. All publications, patents, and patent applications are hereby incorporated by reference to the same extent as if each individual publication or patent application was specifically and individually indicated to be incorporated by reference.

What is claimed is:

1. An isolated polynucleotide comprising a member selected from the group consisting of:
a) a polynucleotide having at least 75% sequence identity to a polynucleotide selected from the group consisting of SEQ ID NO: 1, 9, 11, 13, 15, 17, 19, 21, 23, 25, 27, 31, 33, 37, 39, 40, 41, 42, and 43;
b) a polynucleotide having at least 80% sequence identity to SEQ ID NOS 29;
c) a polynucleotide that hybridizes under high stringency conditions to a polynucleotide selected from the group consisting of SEQ ID NO: 1, 9, 11, 13, 15, 17, 21, 23, 25, 27, 29, 31, 33, 37, 39, 40, 41, 42, and 43; and
d) a polynucleotide complementary to a polynucleotide of (a) through (c)

2. A vector comprising the polynucleotide of claim 1.

3. A recombinant expression cassette comprising the polynucleotide of claim 1 operably linked to a promoter, wherein the nucleic acid is in sense or antisense orientation.

4. The recombinant expression cassette of claim 3, wherein the promoter is selected from the group consisting of a tissue-preferred promoter, a constitutive promoter, and an inducible promoter.

5. A host cell comprising the recombinant expression cassette of claim 3.

6. A transgenic plant comprising the recombinant expression cassette of claim 3.

7. The transgenic plant of claim 6, wherein the plant is maize, soybean, sunflower, sorghum, canola, wheat, alfalfa, cotton, rice, barley, and millet.

8. A transgenic seed from the transgenic plant of claim 6.

9. An isolated protein comprising a member selected from the group consisting of:
a) a polypeptide comprising at least 75% sequence identity to a polypeptide selected from the group consisting of SEQ ID NO: 2, 10, 12, 14, 16, 18, 20, 22, 24, 26, 28, 32, 34, and 38;
b) a polypeptide comprising at least 80% sequence identity to SEQ ID NO: 30;
c) a polypeptide encoded by a polynucleotide selected from the group consisting of SEQ ID NO: 1, 9, 11, 13, 15, 17, 19, 21, 23, 25, 27, 29, 31, 33, and 37; and
d) a polypeptide characterized by a polypeptide selected from the group consisting of SEQ ID NO: 2, 10, 12, 14, 16, 18, 20, 22, 24, 26, 28, 30, 32, 34, and 38.

10. A method of modulating the level of WRKY protein in a plant, comprising:
a) introducing into a plant cell a recombinant expression cassette comprising a WRKY polynucleotide of claim 1 operably linked to a promoter;
b) culturing the plant cell under plant growing conditions to produce a regenerated plant; and
c) inducing expression of said polynucleotide for a time sufficient to modulate the WRKY protein in said plant.

11. The method of claim 10, wherein the plant is maize, soybean, sunflower, sorghum, canola, wheat, alfalfa, cotton, rice, barley, and millet.

12. An isolated polynucleotide comprising a polynucleotide having at least 90% sequence identity to a polynucleotide selected from the group consisting of SEQ ID NO: 1, 9, 11, 13, 15, 17, 19, 21, 23, 25, 27, 29, 31, 33, 37, 39, 40, 41, 42, and 43.

13. A vector comprising the polynucleotide of claim 12.

14. A recombinant expression cassette, comprising the polynucleotide of claim 12, operably linked to a promoter, wherein the nucleic acid is in sense or antisense orientation.

15. The recombinant expression cassette of claim 14, wherein the promoter is selected from the group consisting of a tissue-preferred promoter, a constitutive promoter, and an inducible promoter.

16. A host cell comprising the recombinant expression cassette of claim 14.

17. A transgenic plant comprising the recombinant expression cassette of claim 14.

18. The transgenic plant of claim 17, wherein the plant is maize, soybean, sunflower, sorghum, canola, wheat, alfalfa, cotton, rice, barley, and millet.

19. A transgenic seed from the transgenic plant of claim 17.

20. A method of modulating the level of WRKY protein in a plant, comprising:
a) introducing into a plant cell a recombinant expression cassette comprising the polynucleotide of claim 12 operably linked to a promoter;
b) culturing the plant cell under plant growing conditions to produce a regenerated plant; and
c) inducing expression of said polynucleotide for a time sufficient to modulate WRKY protein in said plant.

21. An isolated polynucleotide comprising a member selected from the group consisting of:
a) a polynucleotide that encodes a polypeptide selected from the group consisting of SEQ ID NO: 2, 10, 12, 14, 16, 18, 20, 22, 24, 26, 28, 30, 32, 34, and 38; and
b) a polynucleotide selected from the group consisting of SEQ ID NO: 1, 9, 11, 13, 15, 17, 19, 21, 23, 25, 27, 29, 31, 33, 37, 39, 40, 41, 42, and 43.

22. A vector comprising the polynucleotide of claim 21.

23. A recombinant expression cassette comprising the polynucleotide of claim 21 operably linked to a promoter, wherein the polynucleotide is in sense or antisense orientation.

24. The recombinant expression cassette of claim 23, wherein the promoter is selected from the group consisting of a tissue-preferred promoter, a constitutive promoter, and an inducible promoter.

25. A host cell comprising the recombinant expression cassette of claim 23.

26. A transgenic plant comprising the recombinant expression cassette of claim 23.

27. The transgenic plant of claim 26, wherein the plant is maize, soybean, sunflower, sorghum, canola, wheat, alfalfa, cotton, rice, barley, and millet.

28. A transgenic seed from the transgenic plant of claim 26.

29. A method of modulating the level of WRKY protein in a plant, comprising:
a) introducing into a plant cell a recombinant expression cassette comprising the polynucleotide of claim 21 operably linked to a promoter;
b) culturing the plant cell under plant growing conditions to produce a regenerated plant; and
c) inducing expression of said polynucleotide for a time sufficient to modulate WRKY protein in said plant.

30. An isolated transcriptional region that is capable of driving transcription in a plant, wherein the transcriptional region comprises a polynucleotide selected from:
a) a polynucleotide driving expression of a WRKY polynucleotide, wherein the WRKY polynucleotide is a polynucleotide having 90% identity to a polynucleotide selected from the group consisting of SEQ ID NOS: 1, 9, 11, 13, 15, 17, 19, 21, 23, 25, 27, 29, 31, 33, 37, 39, 40, 41, 42, and 43;
b) a polynucleotide driving expression of a WRKY polynucleotide, wherein the WRKYpolynucleotide is selected from SEQ ID NOS: 1, 9, 11, 13, 15, 17, 19, 21, 23, 25, 27, 29, 31, 33, 37, 39, 40, 41, 42, and 43;
c) a polynucleotide comprising at least 20 contiguous nucleotides of the sequence set forth in SEQ ID NO: 35;
d) a polynucleotide that hybridizes under highly stringent conditions to the sequence set forth in SEQ ID NO: 35; and
e) a polynucleotide having at least 90% identity to SEQ ID NO: 35.

31. A method of regulating transcription of a heterologous nucleic acid comprising the steps of:
a) introducing into a plant cell the polynucleotide of claim 30 operably linked to a heterologous nucleic acid;
b) culturing the plant cell under plant growing conditions to produce a regenerated plant; and
c) inducing expression of the heterologous nucleic acid.

32. A vector comprising the polynucleotide of claim 30.

33. A recombinant expression cassette comprising the polynucleotide of claim 30 operably linked to a heterologous nucleic acid.

34. The recombinant expression cassette of claim 33, wherein expression of the heterologous nucleic acid increases resistance to plant pathogen.

35. A transgenic plant comprising the recombinant expression cassette of claim 33.

36. An isolated transcriptional region that is capable of driving transcription in a plant, wherein the transcriptional region comprises the polynucleotide shown in SEQ ID NO: 35.

37. A method of regulating the SA-dependent SAR response in a plant comprising the steps of:
a) introducing into a plant cell a recombinant expression cassette comprising the polynucleotide of claim 1 operably linked to a promoter;
b) culturing the plant cell under plant growing conditions to produce a regenerated plant; and
c) inducing expression of said polynucleotide for a time sufficient to modulate the SA-dependent SAR response.

38. The method of claim 37, wherein the polynucleotide is shown in SEQ ID NO: 1.

39. The method of claim 38, wherein the polynucleotide is in the antisense orientation.
